(19) United States
(12) Patent Application Publication
(10) Pub. No.: US 2010/0113330 A1
(43) Pub. Date: May 6, 2010

(54) TETRAHYDROPYRIDO[4,3-D]PYRIMIDINONE DERIVATIVES AND METHODS OF USE THEREOF

(75) Inventors: Craig D. Boyle, Branchburg, NJ (US); Bernard R. Neustadt, West Orange, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/594,044

(22) PCT Filed: Apr. 17, 2008
(86) PCT No.: PCT/US08/04997

§ 371 Date: Dec. 2, 2009

Related U.S. Application Data
(60) Provisional application No. 60/925,432, filed on Apr. 20, 2007.
(60) Provisional application No. 60/953,330, filed on Aug. 1, 2007.

(51) Int. Cl. A61K 31/519; A61K 38/28; A61K 38/16; A61P 3/04; A61P 3/10; A61P 3/00; A61P 9/12

(52) U.S. Cl. 514/4; 514/12; 514/264.1

(57) ABSTRACT

The present invention relates to methods of using Tetrahydropyrido[4,3-d]Pyrimidinone Derivatives for treating or preventing obesity, diabetes, a metabolic disorder, a cardiovascular disease or a disorder related to the activity of GPR119 in a patient.

### FIELD OF THE INVENTION

The present invention relates to methods of using Tetrahydropyrido[4,3-d]Pyrimidinone Derivatives for treating or preventing obesity, diabetes, a metabolic disorder, a cardiovascular disease or a disorder related to the activity of G protein-coupled receptor 119 (“GPR119”) in a patient.

### BACKGROUND OF THE INVENTION

Although a number of receptor classes exist in humans, by far the most abundant and therapeutically relevant is represented by the G protein-coupled receptor (GPCR or GPCRs) class. It is estimated that there are some 100,000 genes within the human genome, and of these, approximately 2% or 2,000 genes, are estimated to code for GPCRs. Receptors, including GPCRs, for which the endogenous ligand has been identified are referred to as “known” receptors, while receptors for which the endogenous ligand has not been identified are referred to as “orphan” receptors. GPCRs represent an important area for the development of pharmaceutical products, as evidenced by the fact that pharmaceutical products have been developed from approximately 20 of the 100 known GPCRs. This distinction is not merely semantic, particularly in the case of GPCRs. Thus, the orphan GPCRs are to the pharmaceutical industry what gold was to California in the late 19th century—an opportunity to drive growth, expansion, enhancement and development.

GPCRs share a common structural motif. All these receptors have seven sequences of between 22 to 24 hydrophobic amino acids that form seven alpha helices, each of which spans the membrane (each span is identified by number, i.e., transmembrane-1 (TM-1), transmembrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when an endogenous ligand binds with the receptor (often referred to as “activation” of the receptor), there is a change in the conformation of the intracellular region that allows for coupling between the intracellular region and an intracellular “G-protein.” It has been reported that GPCRs are “promiscuous” with respect to G proteins, i.e., that a GPCR can interact with more than one G protein. See, Kenakin, T., Life Sciences 43:1095 (1988). Although other G proteins exist, currently, Gq, Gs, Gi, and Go are G proteins that have been identified. Endogenous ligand-activated GPCR coupling with the G-protein begins a signaling cascade process (referred to as “signal transduction”). Under normal conditions, signal transduction ultimately results in cellular activation or cellular inhibition. It is thought that the IC-3 loop as well as the carboxy terminus of the receptor interact with the G protein.

Under physiological conditions, GPCRs exist in the cell membrane in equilibrium between two different conformations: an “inactive” state and an “active” state. A receptor in an inactive state is unable to link to the intracellular signaling transduction pathway to produce a biological response. Changing the receptor conformation to the active state allows linkage to the transduction pathway (via the G-protein) and produces a biological response. A receptor can be stabilized in an active state by an endogenous ligand or a compound such as a drug.

Modulation of G-protein coupled receptors has been well-studied for controlling various metabolic disorders. Small molecule modulators of the receptor GPR119, a G-protein coupled-receptor described in, for example, GenBank (see, e.g., accession numbers XM.sub.--066873 and AY288416), have been shown to be useful for treating or preventing certain metabolic disorders. GPR119 is a G protein-coupled receptor that is selectively expressed on pancreatic beta cells. GPR119 activation leads to elevation of a level of intracellular cAMP, consistent with GPR119 being coupled to Gs. Agonists to GPR119 stimulate glucose-dependent insulin secretion in vitro and lower an elevated blood glucose level in vivo. See, e.g., International Publication Nos. WO 04/065380 and WO 04/076413, and European Patent Application No. EP 1338651, the disclosure of each of which is herein incorporated by reference in its entirety.

U.S. Pat. No. 7,132,426 discloses pyrazolo[3,4-d]pyrimidine ethers and related compounds as modulators of the GPR119 receptor that are useful for the treatment of various metabolic-related disorders such as type I diabetes, type II diabetes, inadequate glucose tolerance, insulin resistance, hyperglycemia, hyperlipidemia, hypertriglyceridemia, hypercholesterolemia, dyslipidemia or syndrome X. The compounds are also reported as being useful for controlling weight gain, controlling food intake, and inducing satiety in mammals. The promising nature of these GPR119 modulators indicates a need in the art for additional small molecule GPR119 modulators with improved efficacy and safety profiles. This invention addresses that need.

### SUMMARY OF THE INVENTION

In one aspect, the present invention provides methods for treating or preventing obesity, diabetes, metabolic syndrome, a cardiovascular disease or a disorder related to the activity of GPR119 (each being a “Condition”) in a patient, the methods comprising administering to the patient an effective amount of one or more compounds of Formula (I):

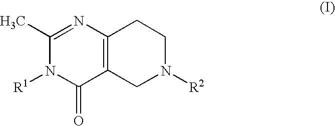

or a pharmaceutically acceptable salt, solvate, ester or prodrug thereof, wherein R¹ and R² are denoted using an “X” as set forth below in Tables A-D, and R¹ and R² are defined below in Tables E and F, respectively.

TABLE A

| | R¹ | | | | | | | | | |
|---|---|---|---|---|---|---|---|---|---|---|
| R² | 1 | 2 | 3 | 4 | 5 | 6 | 7 | 8 | 9 | 10 |
| 1 | X | X | X | X | X | X | X | X | X | X |
| 2 | X | X | X | X | X | X | X | X | X | X |
| 3 | X | X | X | X | X | X | X | X | X | X |
| 4 | X | X | X | X | X | X | X | X | X | X |
| 5 | X | X | X | X | X | X | X | X | X | X |
| 6 | X | X | X | X | X | X | X | X | X | X |
| 7 | X | X | X | X | X | X | X | X | X | X |
| 8 | X | X | X | X | X | X | X | X | X | X |
| 9 | X | X | X | X | X | X | X | X | X | X |
| 10 | X | X | X | X | X | X | X | X | X | X |
| 11 | X | X | X | X | X | X | X | X | X | X |
| 12 | X | X | X | X | X | X | X | X | X | X |
| 13 | X | X | X | X | X | X | X | X | X | X |
| 14 | X | X | X | X | X | X | X | X | X | X |
| 15 | X | X | X | X | X | X | X | X | X | X |
| 16 | X | X | X | X | X | X | X | X | X | X |
| 17 | X | X | X | X | X | X | X | X | X | X |
| 18 | X | X | X | X | X | X | X | X | X | X |
| 19 | X | X | X | X | X | X | X | X | X | X |
| 20 | X | X | X | X | X | X | X | X | X | X |
| 21 | X | X | X | X | X | X | X | X | X | X |
| 22 | X | X | X | X | X | X | X | X | X | X |
| 23 | X | X | X | X | X | X | X | X | X | X |
| 24 | X | X | X | X | X | X | X | X | X | X |
| 25 | X | X | X | X | X | X | X | X | X | X |
| 26 | X | X | X | X | X | X | X | X | X | X |
| 27 | X | X | X | X | X | X | X | X | X | X |
| 28 | X | X | X | X | X | X | X | X | X | X |
| 29 | X | X | X | X | X | X | X | X | X | X |
| 30 | X | X | X | X | X | X | X | X | X | X |
| 31 | X | X | X | X | X | X | X | X | X | X |
| 32 | X | X | X | X | X | X | X | X | X | X |
| 33 | X | X | X | X | X | X | X | X | X | X |
| 34 | X | X | X | X | X | X | X | X | X | X |
| 35 | X | X | X | X | X | X | X | X | X | X |
| 36 | X | X | X | X | X | X | X | X | X | X |
| 37 | X | X | X | X | X | X | X | X | X | X |
| 38 | X | X | X | X | X | X | X | X | X | X |
| 39 | X | X | X | X | X | X | X | X | X | X |
| 40 | X | X | X | X | X | X | X | X | X | X |
| 41 | X | X | X | X | X | X | X | X | X | X |
| 42 | X | X | X | X | X | | X | X | X | X |
| 43 | X | X | X | X | X | | | | | |
| 44 | X | X | X | X | X | X | X | X | X | X |
| 45 | X | X | X | X | X | X | | | X | |
| 46 | X | X | X | X | X | X | X | X | X | X |
| 47 | X | X | X | X | X | X | X | X | X | X |
| 48 | X | X | X | X | X | X | X | X | X | X |
| 49 | X | X | X | X | X | X | X | X | X | X |
| 50 | X | X | X | X | X | X | X | X | X | X |
| 51 | X | X | X | X | X | X | X | X | X | X |
| 52 | X | X | X | X | X | X | X | X | X | X |
| 53 | X | X | X | X | X | X | X | X | X | X |
| 54 | X | X | X | X | X | X | X | X | X | X |
| 55 | X | X | X | X | X | X | X | X | X | X |
| 56 | X | X | X | X | X | X | X | X | X | X |
| 57 | X | X | | X | X | X | X | X | X | X |
| 58 | X | X | X | X | X | X | X | X | X | X |
| 59 | X | X | X | X | X | X | X | X | X | X |
| 60 | X | X | X | X | X | X | X | X | X | X |
| 61 | X | X | X | X | | X | X | X | X | X |
| 62 | X | X | X | X | X | X | X | X | X | X |
| 63 | X | X | | X | | X | X | X | X | X |
| 64 | X | X | X | X | X | X | X | X | X | X |
| 65 | X | X | | X | X | X | X | X | X | X |
| 66 | X | X | | X | | X | X | X | X | X |
| 67 | X | X | | X | | X | X | X | X | X |
| 68 | X | X | | X | | X | X | X | X | X |
| 69 | X | X | | X | | X | X | X | X | X |
| 70 | X | X | | X | | X | X | X | X | X |
| 71 | X | X | X | X | X | X | X | X | X | X |
| 72 | X | X | X | X | | X | X | X | X | X |
| 73 | X | X | X | X | X | X | X | X | X | X |
| 74 | X | X | X | X | X | X | X | X | X | X |
| 75 | X | X | X | X | X | X | X | X | X | X |
| 76 | X | X | X | X | X | X | X | X | X | X |
| 77 | X | X | X | X | X | X | X | X | X | X |
| 78 | X | X | X | X | X | X | X | X | X | X |
| 79 | X | X | X | X | X | X | X | X | X | X |
| 80 | X | X | X | X | X | X | X | X | X | X |
| 81 | | X | X | X | X | | | | | |
| 82 | | X | X | X | X | | | | | |
| 83 | | X | X | X | X | | | | | |
| 84 | | X | X | X | X | X | X | X | | X |
| 85 | | X | X | X | X | | | | | |
| 86 | | X | X | X | X | X | X | X | X | X |
| 87 | | X | X | X | X | | | | | |
| 88 | | X | X | X | X | | | | | |
| 89 | X | X | | X | | X | X | | X | X |
| 90 | X | X | | X | | X | X | X | X | X |
| 91 | X | X | | X | | X | X | X | X | X |
| 92 | X | X | | X | | X | X | X | X | X |
| 93 | X | X | X | X | | X | X | X | X | X |
| 94 | X | X | | X | | X | X | | X | X |
| 95 | X | X | | X | | X | X | X | X | X |
| 96 | X | X | | X | | X | X | X | X | X |
| 97 | X | X | X | X | | X | X | | X | X |
| 98 | X | X | | X | | X | X | | X | X |
| 99 | X | X | X | X | | X | X | X | X | X |
| 100 | X | X | X | X | | X | X | X | X | X |
| 101 | X | X | X | X | | X | X | X | X | X |
| 102 | X | X | X | X | | X | X | X | X | X |
| 103 | X | X | X | X | | X | X | X | X | X |
| 104 | X | X | X | X | | X | X | X | X | X |
| 105 | X | X | X | X | | X | X | X | X | X |
| 106 | X | X | X | X | | X | X | X | X | X |
| 107 | X | X | X | X | | X | X | X | X | X |
| 108 | X | X | X | X | | X | X | X | X | X |
| 109 | X | X | X | X | | X | X | X | X | X |
| 110 | X | X | X | X | | X | X | X | X | X |
| 111 | X | X | X | X | | X | X | | X | X |
| 112 | X | X | X | X | | X | X | X | X | X |
| 113 | X | X | X | X | | X | X | X | X | X |
| 114 | X | X | X | X | | X | X | X | X | X |
| 115 | X | X | X | X | | X | X | X | X | X |
| 116 | X | X | X | X | | X | X | X | X | X |
| 117 | X | X | X | X | | X | X | X | X | X |
| 118 | X | X | X | X | | X | X | X | X | X |
| 119 | X | X | X | X | | X | X | X | X | X |
| 120 | X | X | X | X | | X | X | X | X | X |
| 121 | X | X | X | X | | X | X | X | X | X |
| 122 | X | X | X | X | | X | X | X | X | X |
| 123 | X | X | X | X | | X | X | X | X | X |
| 124 | X | X | X | X | | X | X | | X | X |
| 125 | X | X | X | X | | X | X | | X | X |
| 126 | X | X | X | X | | X | X | X | X | X |
| 127 | X | X | X | X | | X | X | X | X | X |
| 128 | X | X | X | X | | X | X | X | X | X |
| 129 | X | X | X | X | | X | X | X | X | X |
| 130 | X | X | X | X | | X | X | | X | X |
| 131 | X | X | X | X | | X | X | X | X | X |
| 132 | X | X | X | X | | X | X | X | X | X |
| 133 | X | X | X | X | X | X | X | X | X | X |
| 134 | X | X | X | X | X | X | X | X | X | X |
| 135 | X | X | X | X | X | X | X | X | X | X |
| 136 | X | X | X | X | X | X | X | X | X | X |
| 137 | X | X | X | X | X | X | X | X | X | X |
| 138 | X | X | X | X | X | X | X | X | X | X |
| 139 | X | X | X | X | X | X | X | X | X | X |
| 140 | X | X | X | X | X | X | X | X | X | X |
| 141 | X | X | X | X | X | X | X | X | X | X |
| 142 | X | X | X | X | X | X | X | X | X | X |
| 143 | X | X | X | X | X | X | X | X | X | X |
| 144 | X | X | X | X | X | X | X | X | X | X |
| 145 | X | X | X | X | X | X | X | X | X | X |
| 146 | X | X | X | X | X | X | X | X | X | X |
| 147 | X | X | X | | X | X | X | X | X | X |
| 148 | X | X | X | X | X | X | X | X | X | X |
| 149 | X | X | X | X | X | X | X | X | X | X |
| 150 | X | X | X | X | X | X | X | X | X | X |
| 151 | X | X | X | X | X | X | X | X | X | X |
| 152 | X | X | X | X | X | X | X | X | X | X |
| 153 | X | X | X | X | X | X | X | X | X | X |
| 154 | X | X | X | X | X | X | X | X | X | X |
| 155 | X | X | X | X | X | X | X | X | X | X |
| 156 | X | X | X | X | X | X | X | X | X | X |
| 157 | X | X | X | X | X | X | X | X | X | X |
| 158 | X | X | X | X | X | X | X | X | X | X |
| 159 | X | X | X | X | X | X | X | X | X | X |
| 160 | X | X | X | X | X | X | X | X | X | X |
| 161 | X | X | X | X | X | X | X | X | X | X |
| 162 | X | X | X | X | X | X | X | X | X | X |
| 163 | X | X | | X | X | X | X | X | X | X |
| 164 | X | X | | X | X | X | X | X | X | X |
| 165 | X | X | X | X | X | X | X | X | X | X |
| 166 | X | X | | X | X | X | X | X | X | X |
| 167 | X | X | X | X | X | X | X | X | X | X |
| 168 | X | X | | X | X | X | X | X | X | X |
| 169 | X | X | X | X | X | X | X | X | X | X |
| 170 | X | X | X | X | X | X | X | X | X | X |
| 171 | X | X | | X | X | X | X | X | X | X |
| 172 | X | X | | X | X | X | X | X | X | X |
| 173 | X | X | X | X | | X | X | X | X | X |
| 174 | X | X | X | X | | X | X | X | X | X |
| 175 | X | X | X | X | | X | X | X | X | X |
| 176 | X | X | | X | | X | X | X | X | X |
| 177 | | X | X | X | X | X | X | X | | X |
| 178 | | X | X | X | X | X | X | X | X | X |
| 179 | | X | | X | X | X | X | X | | |
| 180 | | X | X | X | X | | X | X | | |
| 181 | | X | X | X | X | X | | X | | |
| 182 | | X | X | X | X | X | X | X | X | X |
| 183 | | X | X | X | | X | | | | X |
| 184 | | X | X | X | X | | X | X | | X |
| 185 | | X | X | | X | | X | | | |
| 186 | | X | X | X | X | | | | | |
| 187 | | X | | | | X | X | X | X | |
| 188 | | X | X | X | | | X | X | | |
| 189 | | X | X | X | X | X | | X | | X |
| 190 | | X | X | X | X | X | X | X | X | X |
| 191 | | X | | X | X | X | X | X | X | X |
| 192 | | X | X | | X | | X | X | | |
| 193 | | X | X | X | X | | | | | |
| 194 | | X | | X | X | | X | X | | X |
| 195 | | X | X | X | X | | | | | |
| 196 | | X | X | X | X | X | | X | | X |
| 197 | | X | X | X | | X | X | X | | |
| 198 | | X | X | X | X | X | X | X | X | X |
| 199 | | X | X | X | X | X | X | X | | |
| 200 | | X | X | | | X | | X | | X |
| 201 | | X | | X | X | X | X | X | | X |
| 202 | | X | X | X | | X | X | X | | |
| 203 | | X | X | X | | X | | | | |
| 204 | | X | | | X | X | X | X | X | |
| 205 | | X | | X | X | X | | X | | |
| 206 | | X | X | X | X | X | X | X | X | |
| 207 | | X | X | X | X | | | | | |
| 208 | | X | X | X | X | X | X | X | | |
| 209 | | X | X | X | X | | | | | |
| 210 | | | X | X | X | X | X | X | X | X |
| 211 | | | X | | | X | X | X | | |
| 212 | | | | | X | X | X | X | | |
| 213 | | | | | | X | X | | | |
| 214 | | | | | | | | X | | |
| 215 | | | | | | | | | | |
| 216 | | X | | X | X | X | X | X | X | X |
| 217 | | X | | X | X | X | X | X | X | X |
| 218 | | X | X | X | X | X | X | X | | X |
| 219 | | X | X | X | X | X | X | X | X | X |
| 220 | | X | | | | X | | | | X |
| 221 | | X | | X | X | X | X | X | | X |
| 222 | | X | | | | | | X | | X |
| 223 | | X | | | X | X | | | | X |
| 224 | | X | X | X | X | X | X | X | | X |
| 225 | | X | X | X | X | | | | X | X |
| 226 | | X | | | X | X | X | X | | X |
| 227 | | X | | X | X | X | X | X | | X |
| 228 | | | X | X | X | | X | X | | X |
| 229 | | | X | X | X | | | X | | |
| 230 | | | | | X | X | X | X | X | X |
| 231 | | | | | X | X | X | X | | X |
| 232 | | | | | | X | | | | X |
| 233 | | | | | | X | | | | X |
| 234 | | | | | | | X | X | X | |
| 235 | X | X | X | X | X | X | X | X | | X |
| 236 | X | X | X | X | X | X | X | X | X | X |
| 237 | X | X | X | X | X | | X | X | X | X |
| 238 | X | X | X | X | X | X | X | X | X | X |
| 239 | X | X | X | X | X | X | X | X | X | X |
| 240 | X | X | X | X | X | X | X | X | X | X |
| 241 | X | X | X | X | X | X | X | X | X | X |
| 242 | X | X | X | X | X | X | X | X | X | X |
| 243 | X | X | X | X | X | X | X | X | X | X |
| 244 | X | X | X | X | | X | X | X | | X |
| 245 | X | X | X | X | X | X | X | X | X | X |
| 246 | X | X | | X | X | X | X | X | | X |
| 247 | X | X | X | X | X | X | X | X | X | X |
| 248 | X | X | X | X | X | X | X | X | X | X |
| 249 | X | X | X | X | X | X | X | X | X | X |
| 250 | X | X | X | X | X | X | X | X | | X |
| 251 | X | X | X | X | X | X | X | X | X | X |
| 252 | X | X | X | X | X | X | X | X | X | X |
| 253 | X | X | X | X | X | X | X | X | X | X |
| 254 | X | X | X | X | X | X | X | X | X | X |
| 255 | X | X | X | X | X | X | X | X | X | X |
| 256 | X | X | X | X | X | X | X | X | X | X |
| 257 | X | X | X | X | X | X | X | X | X | X |
| 258 | X | X | X | X | | X | X | X | X | X |
| 259 | X | X | | X | X | X | X | X | X | X |
| 260 | X | X | X | X | X | | X | X | X | X |
| 261 | X | X | X | | X | | | X | | X |
| 262 | X | X | | | X | X | | X | | X |
| 263 | X | X | X | X | X | X | | X | X | X |
| 264 | X | X | | X | X | X | X | X | X | X |
| 265 | X | X | X | X | X | X | X | X | | X |
| 266 | X | X | X | X | X | X | X | X | | X |
| 267 | X | X | X | X | X | X | X | X | X | X |
| 268 | X | X | X | X | X | X | X | X | X | X |
| 269 | | X | X | X | X | X | X | X | X | X |
| 270 | | X | X | X | X | X | X | X | X | X |
| 271 | | X | X | X | | X | X | X | X | X |
| 272 | | X | | | X | | | X | X | X |
| 273 | | X | X | X | X | X | X | X | X | X |
| 274 | | X | X | X | X | X | X | X | X | X |
| 275 | | X | X | X | X | X | X | X | | X |
| 276 | | X | X | X | X | X | X | X | X | X |
| 277 | | X | X | X | | X | X | X | | X |
| 278 | | | | X | X | X | | X | X | X |

TABLE B

| | R¹ | | | | | | | | | |
|---|---|---|---|---|---|---|---|---|---|---|
| R² | 11 | 12 | 13 | 14 | 15 | 16 | 17 | 18 | 19 | 20 |
| 1 | X | X | X | X | X | X | X | X | X | X |
| 2 | X | X | X | X | X | X | X | X | X | X |
| 3 | X | X | X | X | X | X | X | X | X | X |
| 4 | X | X | X | X | X | X | X | X | X | X |
| 5 | X | X | X | X | | X | X | X | X | X |
| 6 | X | X | X | X | X | X | X | X | X | X |
| 7 | X | X | X | X | X | X | X | X | X | X |
| 8 | X | X | X | X | X | X | X | X | X | X |
| 9 | X | X | X | X | X | X | X | X | X | X |
| 10 | X | X | X | X | X | X | X | X | X | X |
| 11 | X | X | X | X | X | X | X | X | X | X |
| 12 | X | X | X | X | X | X | X | X | X | X |
| 13 | X | X | X | X | X | X | X | | X | X |
| 14 | X | X | X | X | X | X | | X | | X |
| 15 | X | X | X | X | X | X | X | X | X | X |
| 16 | X | X | X | X | X | X | X | X | X | X |
| 17 | X | X | X | X | X | X | X | X | X | X |
| 18 | X | X | X | X | X | X | X | X | X | X |
| 19 | X | X | X | X | X | X | X | X | X | X |
| 20 | X | X | X | X | X | X | X | X | X | X |
| 21 | X | X | X | X | X | X | X | X | X | X |
| 22 | X | X | X | X | X | X | X | X | X | X |
| 23 | X | X | X | X | X | X | X | X | X | X |
| 24 | X | X | X | X | X | X | X | X | X | X |
| 25 | X | X | X | X | X | X | X | X | X | X |
| 26 | X | X | X | X | X | X | X | X | X | X |
| 27 | X | X | X | X | X | X | X | X | X | X |
| 28 | X | X | X | X | X | X | X | X | X | X |
| 29 | X | X | X | X | X | X | X | X | X | X |
| 30 | X | X | X | X | X | X | X | X | X | X |
| 31 | X | X | X | X | X | X | X | X | X | X |
| 32 | X | X | X | X | X | X | X | X | X | X |
| 33 | X | X | X | X | X | X | X | X | X | X |
| 34 | X | X | X | X | X | X | X | X | X | X |
| 35 | X | X | X | X | X | X | X | X | X | X |
| 36 | X | X | X | X | X | X | X | X | X | X |
| 37 | X | X | X | X | X | X | X | X | X | X |
| 38 | X | X | X | X | X | X | X | X | X | X |
| 39 | X | X | X | X | X | X | X | X | X | X |
| 40 | X | X | X | X | X | X | X | X | X | X |
| 41 | X | X | X | X | X | X | X | X | X | X |
| 42 | X | X | X | X | X | X | X | X | X | X |
| 43 | X | X | | | X | X | | | | X |
| 44 | X | X | X | X | X | X | X | X | X | X |
| 45 | | X | | | X | X | X | X | X | X |
| 46 | X | X | X | X | X | X | X | X | X | X |
| 47 | X | X | X | X | X | X | X | X | X | X |
| 48 | | | | X | | X | X | X | | X |
| 49 | X | X | X | X | | X | X | X | X | X |
| 50 | X | X | X | X | X | X | X | X | X | X |
| 51 | X | X | X | X | X | X | X | X | X | X |
| 52 | X | X | X | X | X | X | X | X | X | X |
| 53 | X | | X | X | X | X | X | X | X | |
| 54 | X | X | X | X | X | | X | X | X | |
| 55 | X | X | X | X | X | X | X | X | X | X |
| 56 | X | X | X | X | X | X | X | X | X | X |
| 57 | X | X | X | X | X | X | X | X | X | X |
| 58 | X | X | X | X | X | X | X | X | X | X |
| 59 | X | X | X | X | X | X | X | X | X | X |
| 60 | X | X | X | X | X | X | X | X | X | X |
| 61 | X | X | X | X | X | X | X | X | X | X |
| 62 | X | X | X | X | X | X | X | X | X | X |
| 63 | X | X | X | X | X | X | X | X | X | X |
| 64 | X | X | X | X | X | | X | X | X | |
| 65 | X | X | X | X | X | | X | X | X | |
| 66 | X | X | X | X | X | | X | X | X | |
| 67 | X | X | X | X | X | | X | X | X | |
| 68 | X | X | X | X | X | X | X | X | X | X |
| 69 | X | X | X | X | X | | X | X | X | |
| 70 | X | X | X | X | X | | X | X | X | |
| 71 | X | X | X | X | X | X | X | X | X | X |
| 72 | X | X | X | X | X | | X | X | X | X |
| 73 | X | X | X | X | X | X | X | X | X | X |
| 74 | X | X | X | X | X | X | X | X | X | X |
| 75 | X | X | X | X | X | X | X | X | X | X |
| 76 | X | X | X | X | X | X | X | X | X | X |
| 77 | X | X | X | X | X | X | X | X | X | X |
| 78 | X | X | X | X | X | X | X | X | X | X |
| 79 | X | X | X | X | X | X | X | X | X | X |
| 80 | X | X | X | X | X | X | X | X | X | X |
| 81 | | | | | | X | | | | X |
| 82 | | | | | | X | | | X | X |
| 83 | | | X | | | X | X | X | X | X |
| 84 | X | X | X | X | | X | | | | X |
| 85 | | | | | X | X | | | | X |
| 86 | X | X | X | X | X | X | X | X | X | X |
| 87 | | | | | X | X | | | | X |
| 88 | | | | | | X | | | | X |
| 89 | X | X | X | X | X | X | X | X | X | X |
| 90 | X | X | X | X | X | X | X | X | X | X |
| 91 | X | X | X | X | X | X | X | X | X | X |
| 92 | X | X | X | X | X | X | X | X | X | X |
| 93 | X | X | X | X | X | X | X | X | X | X |
| 94 | X | X | X | X | X | X | X | X | | X |
| 95 | X | X | X | X | X | X | X | X | X | X |
| 96 | X | X | X | X | X | X | X | X | X | X |
| 97 | X | X | X | X | X | X | X | X | X | X |
| 98 | X | X | X | X | X | X | X | X | | X |
| 99 | X | X | X | X | X | X | X | X | X | X |
| 100 | X | X | X | X | X | X | X | X | X | X |
| 101 | X | X | X | X | X | X | X | X | X | X |
| 102 | X | X | X | X | X | X | X | X | X | X |
| 103 | X | X | X | X | X | X | X | X | X | X |
| 104 | X | X | X | X | X | X | X | X | X | X |
| 105 | X | X | X | X | X | X | X | X | X | X |
| 106 | X | X | X | X | X | X | X | X | X | X |
| 107 | X | X | X | X | X | X | X | X | X | X |
| 108 | X | X | X | X | X | X | X | X | X | X |
| 109 | X | X | X | X | X | X | X | X | X | X |
| 110 | X | X | X | X | X | X | X | X | X | X |
| 111 | X | | X | X | X | X | X | X | | X |
| 112 | X | X | X | X | X | X | X | X | X | X |
| 113 | X | X | X | X | X | X | X | X | X | X |
| 114 | X | X | X | X | X | X | X | X | X | X |
| 115 | X | X | X | X | X | X | X | X | X | X |
| 116 | X | X | X | X | X | X | X | X | X | X |
| 117 | X | X | X | X | X | X | X | X | X | X |
| 118 | X | X | X | X | X | X | X | X | X | X |
| 119 | X | X | X | X | X | X | X | X | X | X |
| 120 | X | X | X | X | X | X | X | X | X | X |
| 121 | X | X | X | X | X | X | X | X | X | X |
| 122 | X | X | X | X | X | X | X | X | X | X |
| 123 | X | X | X | X | X | X | X | X | X | X |
| 124 | X | X | X | X | X | X | X | | X | X |
| 125 | X | | X | X | X | X | X | X | | X |
| 126 | X | X | X | X | X | X | X | X | X | X |
| 127 | X | | X | X | X | X | X | X | X | X |
| 128 | X | | X | X | X | X | X | X | X | X |
| 129 | X | | X | X | X | X | X | | X | |
| 130 | X | | X | X | X | X | X | | | |
| 131 | X | | X | X | X | X | X | X | X | X |
| 132 | X | | X | X | X | X | X | X | X | X |
| 133 | X | X | X | X | X | X | X | X | X | X |
| 134 | X | X | X | X | X | X | X | X | X | X |
| 135 | X | X | X | X | X | X | X | X | X | X |
| 136 | X | X | X | X | X | X | X | X | X | X |
| 137 | X | X | X | X | X | X | X | X | X | X |
| 138 | X | X | X | X | X | X | X | X | X | X |
| 139 | X | X | X | X | X | X | X | X | X | X |
| 140 | X | X | X | X | X | X | X | X | X | X |
| 141 | X | X | X | X | X | X | X | X | X | X |
| 142 | X | X | X | X | X | X | X | X | X | X |
| 143 | X | X | X | X | X | X | X | X | X | X |
| 144 | X | X | X | X | X | X | X | X | X | X |
| 145 | X | X | X | X | X | X | X | X | X | X |
| 146 | X | X | X | X | X | X | X | X | X | X |
| 147 | X | X | X | X | X | X | X | X | X | X |
| 148 | X | X | X | X | X | X | X | X | X | X |
| 149 | X | X | X | X | X | X | X | X | X | X |
| 150 | X | X | X | X | X | X | X | X | X | X |
| 151 | X | X | X | X | X | X | X | X | X | X |
| 152 | X | X | X | X | X | X | X | X | X | X |
| 153 | X | X | X | X | X | X | X | X | X | X |
| 154 | X | X | X | X | X | X | X | X | X | X |
| 155 | X | X | X | X | X | X | X | X | X | X |
| 156 | X | X | X | X | X | X | X | X | X | X |
| 157 | X | X | X | X | X | X | X | X | X | X |
| 158 | X | X | X | X | X | X | X | X | X | X |
| 159 | X | X | X | X | X | X | X | X | X | X |
| 160 | X | X | X | X | X | X | X | X | X | X |
| 161 | X | X | X | X | X | X | X | X | X | X |
| 162 | X | X | X | X | X | X | X | X | X | X |
| 163 | X | X | X | X | X | X | X | X | X | X |
| 164 | X | X | X | X | X | X | X | X | X | X |
| 165 | X | X | X | X | X | X | X | X | X | X |
| 166 | X | X | X | X | X | X | X | X | X | X |
| 167 | X | X | X | X | X | X | X | X | X | X |
| 168 | X | X | X | X | X | X | X | X | X | X |
| 169 | X | X | X | X | X | X | X | X | X | X |
| 170 | X | X | X | X | X | X | X | X | X | X |
| 171 | X | X | X | X | X | X | X | X | X | X |
| 172 | X | X | X | X | X | X | X | X | X | X |
| 173 | X | X | X | X | X | X | X | X | X | X |
| 174 | X | X | X | X | X | X | X | X | X | X |
| 175 | X | X | X | X | X | X | X | X | X | X |
| 176 | X | X | X | X | X | X | X | X | X | X |
| 177 | X | | X | | | | | X | X | X |
| 178 | X | | X | X | | X | X | X | X | X |
| 179 | X | X | X | | | X | X | X | X | X |
| 180 | | X | | X | X | | | X | | X |
| 181 | | X | X | X | | X | X | X | X | X |
| 182 | X | X | X | X | X | | X | X | X | X |
| 183 | | | X | | | | | X | X | X |
| 184 | X | X | | X | X | | | X | X | X |
| 185 | | | | | | | | | | |
| 186 | | X | | | | | | X | | |
| 187 | | | X | X | | X | X | X | X | X |
| 188 | | | X | | | | | X | X | |
| 189 | | X | X | | X | X | X | X | X | X |
| 190 | X | | | | | | | X | X | X |
| 191 | X | X | X | X | X | | X | X | X | X |
| 192 | X | X | | | | | | | X | |
| 193 | X | X | X | X | | | X | X | X | X |
| 194 | X | | X | X | X | | | X | X | X |
| 195 | | X | | | X | | | | | X |
| 196 | X | X | X | X | X | | | X | X | X |
| 197 | | X | X | X | X | X | X | X | X | X |
| 198 | X | X | | X | X | | X | X | X | X |
| 199 | | X | | X | | X | | X | X | X |
| 200 | X | | | X | | | | X | X | X |
| 201 | X | X | | X | X | X | X | X | X | X |
| 202 | | X | | X | | | X | X | X | X |
| 203 | X | | | | | | | X | X | X |
| 204 | X | X | | X | X | | X | X | X | |
| 205 | | X | | X | X | X | | X | X | X |
| 206 | X | X | X | X | X | | X | X | X | X |
| 207 | X | | | | | | | X | X | X |
| 208 | | X | | X | X | X | X | | X | X |
| 209 | X | | | | | | | | X | X |
| 210 | X | X | X | X | X | X | X | X | X | X |
| 211 | | | | | | | X | X | X | X |
| 212 | X | X | | X | X | X | X | X | X | X |
| 213 | | | | X | X | X | X | X | X | X |
| 214 | | X | | | X | | | X | X | |
| 215 | | | X | | | | | | | X |
| 216 | X | X | X | X | X | X | X | X | X | X |
| 217 | | X | X | X | X | X | X | X | X | X |
| 218 | X | X | X | X | X | X | X | X | X | X |
| 219 | X | X | X | X | X | X | X | | X | X |
| 220 | | X | X | X | | X | X | X | X | X |
| 221 | X | | X | X | X | | | X | | X |
| 222 | X | X | X | | | | | X | | |
| 223 | | | X | | X | X | X | X | X | X |
| 224 | | | X | X | | X | X | X | X | X |
| 225 | X | | X | | X | | X | X | | X |
| 226 | X | | X | X | | | X | X | X | X |
| 227 | X | X | X | X | X | X | X | X | X | X |
| 228 | X | | X | | X | | X | X | | X |
| 229 | | | | X | | | | | | |
| 230 | X | | X | X | X | X | X | X | X | X |
| 231 | | X | X | X | X | X | | X | X | X |
| 232 | | | X | X | X | | | X | | |
| 233 | | X | X | X | | | X | X | X | X |
| 234 | | X | X | X | X | | X | X | X | X |
| 235 | X | X | | X | X | X | X | X | X | X |
| 236 | X | X | X | X | X | X | X | X | X | X |
| 237 | X | X | X | X | X | X | X | X | X | X |
| 238 | X | | X | | X | X | X | X | X | X |
| 239 | X | X | X | X | X | X | X | X | X | X |
| 240 | X | X | X | X | X | X | X | X | X | X |
| 241 | X | X | X | X | X | X | X | X | X | X |
| 242 | X | X | | X | | X | X | X | X | X |
| 243 | X | X | X | X | X | X | X | X | X | X |
| 244 | X | X | X | X | | X | X | | X | X |
| 245 | X | X | X | X | X | X | X | X | X | X |
| 246 | X | | X | X | X | X | | X | | X |
| 247 | X | X | X | X | X | X | X | X | X | X |
| 248 | X | X | X | X | X | X | X | X | X | X |
| 249 | X | X | X | X | X | X | X | X | X | X |
| 250 | X | X | | X | X | X | X | | X | X |
| 251 | X | X | X | X | X | X | X | X | X | X |
| 252 | X | X | X | X | X | X | X | X | X | X |
| 253 | | | X | | X | X | X | X | | X |
| 254 | X | X | X | X | X | X | X | X | X | X |
| 255 | X | X | X | X | X | X | X | X | X | X |
| 256 | X | X | X | X | X | X | X | X | X | X |
| 257 | X | X | | X | | X | X | X | X | X |
| 258 | X | X | X | X | X | X | X | X | X | X |
| 259 | X | X | X | X | X | X | X | X | X | X |
| 260 | X | X | X | X | X | X | X | X | X | X |
| 261 | | X | X | X | X | X | X | X | X | |
| 262 | | X | X | X | X | X | X | X | X | |
| 263 | X | X | | X | X | X | X | X | X | X |
| 264 | X | X | X | X | | X | X | X | X | X |
| 265 | X | X | X | X | X | X | X | X | X | X |
| 266 | X | X | X | X | | X | X | X | X | X |
| 267 | X | X | | X | | X | X | X | X | |
| 268 | X | X | X | X | | X | X | X | X | X |
| 269 | X | X | X | X | X | X | X | X | X | X |
| 270 | X | X | X | X | X | | X | X | X | X |
| 271 | | X | X | X | | X | X | | X | |
| 272 | X | | X | | | X | | X | | |
| 273 | X | X | X | X | | X | X | X | X | X |
| 274 | | | X | | X | X | X | X | X | X |
| 275 | X | X | X | X | X | X | X | X | X | X |
| 276 | X | X | X | X | X | X | X | X | X | X |
| 277 | X | X | X | X | | X | X | X | X | X |
| 278 | | X | X | X | | X | X | X | X | X |

TABLE C

| | R¹ | | | | | | | | | |
|---|---|---|---|---|---|---|---|---|---|---|
| R² | 21 | 22 | 23 | 24 | 25 | 26 | 27 | 28 | 29 | 30 |
| 1 | X | | X | X | X | X | X | X | X | X |
| 2 | X | X | X | X | X | X | X | X | X | X |
| 3 | X | | X | X | X | X | X | X | X | X |
| 4 | X | X | X | X | X | X | X | X | X | X |
| 5 | X | X | X | X | X | X | X | X | X | X |
| 6 | X | X | | X | X | X | X | X | X | X |
| 7 | X | X | X | X | X | X | X | X | X | X |
| 8 | X | X | X | X | X | X | X | X | X | X |
| 9 | X | X | X | X | X | X | X | X | X | X |
| 10 | X | X | X | X | | X | X | X | X | X |
| 11 | X | X | X | X | X | X | X | X | X | X |
| 12 | X | X | | X | | X | X | X | X | X |
| 13 | X | X | X | X | X | X | X | | X | X |
| 14 | X | X | X | X | X | X | X | | X | X |
| 15 | X | | X | X | X | X | X | X | X | X |
| 16 | X | X | X | X | X | X | X | X | X | X |
| 17 | X | X | X | X | X | X | X | X | X | X |
| 18 | X | X | X | X | X | X | X | X | X | X |
| 19 | X | X | X | X | X | X | X | X | X | X |
| 20 | X | X | X | X | X | X | X | X | X | X |
| 21 | X | X | X | X | X | X | X | X | X | X |
| 22 | X | X | X | X | X | X | X | X | X | X |
| 23 | X | X | X | X | X | X | X | X | X | X |
| 24 | X | X | X | X | X | X | X | X | X | X |
| 25 | X | X | X | X | X | X | X | X | X | X |
| 26 | X | X | X | X | X | X | X | X | X | X |
| 27 | X | X | X | X | X | X | X | X | X | X |
| 28 | X | X | X | X | X | X | | X | X | X |
| 29 | X | X | X | X | X | X | X | X | X | X |
| 30 | X | X | X | X | X | X | X | X | X | X |
| 31 | X | X | X | X | X | X | X | X | X | X |
| 32 | X | X | X | X | X | X | X | X | X | X |
| 33 | X | X | X | X | X | X | X | X | X | X |
| 34 | X | X | X | X | X | X | X | X | X | X |
| 35 | X | X | X | X | X | X | X | X | X | X |
| 36 | X | X | X | X | X | X | X | X | X | X |
| 37 | X | X | X | X | X | X | X | X | X | X |
| 38 | X | X | X | X | X | X | X | X | X | X |
| 39 | X | X | X | X | X | X | X | X | X | X |
| 40 | X | X | X | X | X | X | X | X | X | X |
| 41 | X | X | X | X | X | X | X | X | X | X |
| 42 | X | X | X | X | X | X | X | X | X | X |
| 43 | X | X | X | X | X | X | X | X | X | X |
| 44 | X | X | X | X | X | X | X | X | X | X |
| 45 | X | X | X | X | X | X | | X | X | X |
| 46 | X | X | X | X | X | X | X | X | X | X |
| 47 | X | X | X | X | X | X | X | X | X | X |
| 48 | X | X | X | X | | X | X | | | X |
| 49 | X | X | X | X | X | X | X | X | | X |
| 50 | X | X | X | X | X | X | X | X | X | X |
| 51 | X | X | X | X | X | X | X | X | X | X |
| 52 | X | X | X | X | X | X | X | X | X | X |
| 53 | X | X | X | | X | X | X | X | X | X |
| 54 | X | X | X | | X | X | X | X | X | X |
| 55 | X | X | X | X | X | X | X | X | X | X |
| 56 | X | X | X | X | X | | X | X | X | X |
| 57 | X | X | X | X | X | X | X | X | X | X |
| 58 | X | X | X | X | X | X | X | X | X | X |
| 59 | X | X | X | X | X | X | X | X | X | X |
| 60 | X | X | X | X | X | X | X | X | X | X |
| 61 | X | X | X | X | X | | X | X | X | X |
| 62 | X | X | X | X | X | X | X | X | X | X |
| 63 | | X | X | X | X | X | X | X | X | X |
| 64 | | X | X | X | X | X | X | X | X | X |
| 65 | | X | X | | X | X | X | X | X | X |
| 66 | | X | X | | X | | X | X | X | X |
| 67 | | X | X | | X | | X | X | X | X |
| 68 | X | X | X | X | X | X | X | X | X | X |
| 69 | | X | X | | X | | X | X | X | X |
| 70 | | X | X | | X | | X | X | X | X |
| 71 | X | X | X | X | X | | X | X | X | X |
| 72 | | X | X | X | X | | X | X | X | X |
| 73 | X | X | X | X | X | X | X | X | X | X |
| 74 | X | X | X | X | X | X | X | X | X | X |
| 75 | X | X | X | X | X | X | X | X | X | X |
| 76 | X | X | X | X | X | X | X | X | X | X |
| 77 | X | X | X | X | X | X | X | X | X | X |
| 78 | X | X | X | X | X | X | X | X | X | X |
| 79 | X | X | X | X | X | X | X | X | X | X |
| 80 | X | X | X | X | X | X | X | X | X | X |
| 81 | X | | | X | | X | | | | X |
| 82 | X | | | X | X | X | | | | X |
| 83 | X | X | | X | | X | | X | X | X |
| 84 | X | | | X | | X | X | | | X |
| 85 | X | | | X | | X | | X | | X |
| 86 | X | X | X | X | X | X | X | | X | X |
| 87 | X | | | X | | X | | | | X |
| 88 | X | X | X | | X | X | | X | | X |
| 89 | | X | | X | X | X | X | | X | X |
| 90 | X | X | X | X | X | X | X | | X | X |
| 91 | X | X | X | X | X | X | X | | X | X |
| 92 | X | X | X | X | X | X | X | | X | X |
| 93 | X | X | X | X | X | X | X | | X | X |
| 94 | X | X | X | X | X | X | X | | X | X |
| 95 | X | X | X | X | X | X | X | | X | X |
| 96 | X | X | X | X | X | X | X | | X | X |
| 97 | X | X | X | X | X | X | X | X | X | X |
| 98 | | X | | X | X | X | X | X | X | X |
| 99 | X | X | X | X | X | X | X | X | X | X |
| 100 | X | X | X | X | X | X | X | X | X | X |
| 101 | X | X | X | X | X | X | X | X | X | X |
| 102 | X | X | X | X | X | X | X | X | X | X |
| 103 | X | X | X | X | X | X | X | X | X | X |
| 104 | X | X | X | X | X | X | X | X | X | X |
| 105 | X | X | X | X | X | X | X | X | X | X |
| 106 | X | X | X | X | X | X | X | X | X | X |
| 107 | X | X | X | X | X | X | X | X | X | X |
| 108 | X | X | X | X | X | X | X | X | X | X |
| 109 | X | X | X | X | X | X | X | X | X | X |
| 110 | X | X | X | X | X | X | X | X | X | X |
| 111 | | X | | X | X | X | X | X | X | X |
| 112 | X | X | X | X | X | X | X | X | X | X |
| 113 | X | X | X | X | X | X | X | X | X | X |
| 114 | X | X | X | X | X | X | X | X | X | X |
| 115 | X | X | X | X | X | X | X | X | X | X |
| 116 | X | X | X | X | X | X | X | X | X | X |
| 117 | X | X | X | X | X | X | X | X | X | X |
| 118 | X | X | X | X | X | X | X | X | X | X |
| 119 | X | X | X | X | X | X | X | X | X | X |
| 120 | X | X | X | X | X | X | X | X | X | X |
| 121 | X | X | X | X | X | X | X | X | X | X |
| 122 | X | X | X | X | X | X | X | X | X | X |
| 123 | X | X | X | X | X | X | X | X | X | X |
| 124 | | X | | X | X | X | X | X | X | X |
| 125 | | X | | X | X | X | X | X | X | X |
| 126 | X | X | X | X | X | X | X | X | X | X |
| 127 | X | X | X | X | X | X | X | X | X | X |
| 128 | X | X | X | X | X | X | X | X | X | X |
| 129 | X | | X | X | X | X | X | X | X | X |
| 130 | | | | X | X | X | X | X | X | X |
| 131 | X | X | X | X | X | X | X | X | X | X |
| 132 | X | X | X | X | X | X | X | X | X | X |
| 133 | X | X | X | X | X | X | X | X | X | X |
| 134 | X | X | X | X | X | X | X | X | X | X |
| 135 | X | X | X | X | X | X | X | X | X | X |
| 136 | X | X | X | X | X | X | X | X | X | X |
| 137 | X | X | X | X | X | X | X | X | X | X |
| 138 | X | X | X | X | X | X | X | X | X | X |
| 139 | X | X | X | X | X | X | X | X | X | X |
| 140 | X | X | X | X | X | X | X | X | X | X |
| 141 | X | X | X | X | X | X | X | X | X | X |
| 142 | X | X | X | X | X | X | X | X | X | X |
| 143 | X | X | X | X | X | X | X | X | X | X |
| 144 | X | X | X | X | X | X | X | X | X | X |
| 145 | X | X | X | X | X | X | X | X | X | X |
| 146 | X | X | X | X | X | X | X | X | X | X |
| 147 | X | X | X | X | X | X | X | X | X | X |
| 148 | X | X | X | X | X | X | X | X | X | X |
| 149 | X | X | X | X | X | X | X | X | X | X |
| 150 | X | X | X | X | X | X | X | X | X | X |
| 151 | X | X | X | X | X | X | X | X | X | X |
| 152 | X | X | X | X | X | X | X | X | X | X |
| 153 | X | X | X | X | X | X | X | X | X | X |
| 154 | X | X | X | X | X | X | X | X | X | X |
| 155 | X | X | X | X | X | X | X | X | X | X |
| 156 | X | X | X | X | X | X | X | X | X | X |
| 157 | X | X | X | X | X | X | X | X | X | X |
| 158 | X | X | X | X | X | X | X | X | X | X |
| 159 | X | X | X | X | X | X | X | X | X | X |
| 160 | X | X | X | X | X | X | X | X | X | X |
| 161 | X | X | X | X | X | X | X | X | X | X |
| 162 | X | X | X | X | X | X | X | X | X | X |
| 163 | X | X | X | X | X | X | X | X | X | X |
| 164 | X | X | X | X | X | X | X | X | X | X |
| 165 | X | X | X | X | X | X | X | X | X | X |
| 166 | X | X | X | X | X | X | X | X | X | X |
| 167 | X | X | X | X | X | X | X | X | X | X |
| 168 | X | X | X | X | X | X | X | X | X | X |
| 169 | X | X | X | X | X | X | X | X | X | X |
| 170 | X | X | X | X | X | X | X | X | X | X |
| 171 | X | X | X | X | X | X | X | X | X | X |
| 172 | X | X | X | X | X | X | X | X | X | X |
| 173 | X | X | X | X | X | X | X | X | X | X |
| 174 | X | X | X | X | X | X | X | X | X | X |
| 175 | 7 | X | X | X | X | X | X | X | X | X |
| 176 | X | X | X | X | X | X | X | X | X | X |
| 177 | X | X | X | | X | X | X | X | X | |
| 178 | X | X | X | X | X | | X | X | X | |
| 179 | X | X | X | X | X | X | | X | | |
| 180 | | | | | X | | | | | |
| 181 | | | | | X | | X | X | | |
| 182 | | X | X | X | X | X | X | X | | |
| 183 | | | | | X | | X | X | | |
| 184 | X | X | X | X | X | X | X | X | | |
| 185 | | | | | | | | | | |
| 186 | | | | | | | | | | |
| 187 | | | | | | | X | X | | |
| 188 | | | | X | X | X | | | | |
| 189 | X | X | X | X | X | | X | X | | |
| 190 | X | X | X | X | X | X | X | X | | |
| 191 | X | X | X | X | X | | X | | | |
| 192 | | | | | X | | | | | |
| 193 | X | X | X | | X | | | X | | |
| 194 | X | X | X | | X | | X | X | | |
| 195 | | | X | | X | | | X | | |
| 196 | X | X | X | X | X | | X | X | | |
| 197 | | | | | X | | X | X | | |
| 198 | X | X | X | X | X | | | X | | |
| 199 | X | | X | | X | | | X | | |
| 200 | X | X | X | X | | | | | | |
| 201 | X | X | X | X | X | | X | X | | |
| 202 | | | X | X | X | | | X | | |
| 203 | | | | | | | X | X | | |
| 204 | | | | X | X | X | | | | |
| 205 | | | X | | X | | | | | |
| 206 | X | X | X | X | X | X | X | X | | |
| 207 | | X | | | X | | X | X | | |
| 208 | X | X | X | X | X | X | X | X | | |
| 209 | X | | X | | X | | | | | |
| 210 | X | X | X | X | X | X | X | X | | |
| 211 | | X | | | X | X | | X | | |
| 212 | X | X | | X | X | | | X | | |
| 213 | | | | | X | X | | | | |
| 214 | | | | | | | X | X | | |
| 215 | | | | | | | | | | |
| 216 | X | X | X | X | X | X | X | X | X | X |
| 217 | X | X | X | X | X | X | X | X | X | X |
| 218 | X | X | X | X | X | X | X | X | X | X |
| 219 | X | X | X | X | X | X | X | X | X | X |
| 220 | X | X | X | | X | | X | X | X | X |
| 221 | X | X | | | X | | X | X | X | X |
| 222 | | X | | | | | | X | | |
| 223 | X | X | | | | | X | X | | X |
| 224 | X | X | X | X | X | X | X | X | X | X |
| 225 | | X | | | | | X | X | | X |
| 226 | X | X | X | | X | X | X | X | X | X |
| 227 | X | X | X | X | X | | X | X | X | X |
| 228 | X | X | | X | X | X | X | X | | X |
| 229 | | | X | | | | | | | |
| 230 | X | X | X | | X | X | X | X | X | X |
| 231 | X | X | X | X | | X | X | X | X | X |
| 232 | X | X | X | | | | X | X | | X |
| 233 | X | X | | X | | X | X | X | X | X |
| 234 | | X | X | X | X | X | X | X | | X |
| 235 | X | X | | | X | | X | X | X | X |
| 236 | X | X | | | X | X | X | X | X | X |
| 237 | X | X | | | X | X | X | X | X | X |
| 238 | X | X | | | X | X | X | X | X | X |
| 239 | X | X | | | X | X | X | X | X | X |
| 240 | X | X | | | X | X | X | X | X | X |
| 241 | X | X | | | X | X | X | X | X | X |
| 242 | X | X | | | X | X | X | X | X | X |
| 243 | X | X | | | X | X | X | X | X | X |
| 244 | X | | | | | X | X | X | X | X |
| 245 | X | X | | | X | X | X | X | X | X |
| 246 | X | | | | X | | X | X | X | X |
| 247 | X | X | | | X | X | X | X | X | X |
| 248 | X | X | | | X | X | X | X | X | X |
| 249 | X | X | | | X | X | X | X | X | X |
| 250 | X | X | | | X | X | X | X | X | X |
| 251 | X | X | | | X | X | X | X | X | X |
| 252 | X | X | | | X | X | X | X | X | X |
| 253 | X | X | | | X | X | X | X | X | X |
| 254 | X | X | | | X | X | X | X | X | X |
| 255 | X | X | | | X | X | X | X | X | X |
| 256 | X | X | | | X | X | X | X | X | X |
| 257 | X | X | | | X | X | X | X | X | X |
| 258 | X | X | | | X | X | X | X | X | X |
| 259 | X | X | | | X | X | X | X | X | X |
| 260 | X | X | | | X | X | X | X | X | X |
| 261 | | | | | X | X | X | X | | X |
| 262 | X | X | | | X | X | X | X | | X |
| 263 | X | X | | | X | X | | X | X | X |
| 264 | X | X | | | X | X | X | X | X | X |
| 265 | X | | | | X | X | X | X | X | X |
| 266 | X | | | | X | X | X | X | X | X |
| 267 | X | | | | X | | X | X | X | X |
| 268 | X | X | | | X | X | X | X | X | X |
| 269 | X | X | | | X | X | X | X | X | X |
| 270 | X | X | | | X | X | X | X | X | X |
| 271 | | X | | | | | X | X | X | X |
| 272 | X | | | | X | X | | X | | X |
| 273 | X | X | | | X | X | X | X | X | X |
| 274 | X | | | | X | X | | X | X | X |
| 275 | X | X | | | X | X | X | X | X | X |
| 276 | X | X | | | X | X | X | X | X | X |
| 277 | X | X | | | X | X | X | X | X | X |
| 278 | | X | | | | X | X | X | X | X |

TABLE D

| | R¹ | | | | | | | | | |
|---|---|---|---|---|---|---|---|---|---|---|
| R² | 31 | 32 | 33 | 34 | 35 | 36 | 37 | 38 | 39 | 40 |
| 1 | X | X | X | X | X | | | | | |
| 2 | X | X | X | X | X | | | | | |
| 3 | X | X | X | X | X | | | | | |
| 4 | X | X | X | X | X | | | | | |
| 5 | X | X | X | X | X | | | | | |
| 6 | X | X | X | X | X | | | | | |
| 7 | X | X | X | X | X | | | | | |
| 8 | X | X | X | X | X | | | | | |
| 9 | X | X | X | X | X | | | | | |
| 10 | X | X | X | X | X | | | | | |
| 11 | X | X | X | X | X | | | | | |
| 12 | X | X | X | X | X | | | | | |
| 13 | X | X | X | X | X | | | | | |
| 14 | X | X | X | X | X | | | | | |
| 15 | X | X | X | X | X | | | | | |
| 16 | X | X | X | X | X | | | | | |
| 17 | X | X | X | X | X | | | | | |
| 18 | X | X | X | X | X | | | | | |
| 19 | X | X | X | X | X | | | | | |
| 20 | X | X | X | X | X | | | | | |
| 21 | X | X | X | X | X | | | | | |
| 22 | X | X | X | X | X | | | | | |
| 23 | X | X | X | X | X | | | | | |
| 24 | X | X | X | X | X | | | | | |
| 25 | X | X | X | X | X | | | | | |
| 26 | X | X | X | X | X | | | | | |
| 27 | X | X | X | X | X | | | | | |
| 28 | X | X | X | X | X | | | | | |
| 29 | X | X | X | X | X | | | | | |
| 30 | X | X | X | X | X | | | | | |
| 31 | X | X | X | X | X | | | | | |
| 32 | X | X | X | X | X | | | | | |
| 33 | X | X | X | X | X | | | | | |
| 34 | X | X | X | X | X | | | | | |
| 35 | X | X | X | X | X | | | | | |
| 36 | X | X | X | X | X | | | | | |
| 37 | X | X | X | X | X | | | | | |
| 38 | X | X | X | X | X | | | | | |
| 39 | X | X | X | X | X | | | | | |
| 40 | X | X | X | X | X | | | | | |
| 41 | X | X | X | X | X | | | | | |
| 42 | X | X | X | X | X | | | | | |
| 43 | X | X | X | X | X | | | | | |
| 44 | X | X | X | X | X | | | | | |
| 45 | X | X | X | X | X | | | | | |
| 46 | X | X | X | X | X | | | | | |
| 47 | X | X | X | X | X | | | | | |
| 48 | X | X | X | X | X | | | | | |
| 49 | | X | X | X | X | | | | | |
| 50 | X | X | X | X | X | | | | | |
| 51 | X | X | X | X | X | | | | | |
| 52 | X | X | X | X | X | | | | | |
| 53 | | | X | | | | | | | |
| 54 | | | X | | | | | | | |
| 55 | X | X | X | X | X | | | | | |
| 56 | X | X | X | X | X | | | | | |
| 57 | X | X | X | X | X | | | | | |
| 58 | X | X | X | X | X | | | | | |
| 59 | X | X | X | X | X | | | | | |
| 60 | X | X | X | X | X | | | | | |
| 61 | X | X | X | X | X | | | | | |
| 62 | X | X | X | X | X | | | | | |
| 63 | X | X | X | X | X | | | | | |
| 64 | | | | | | | | | | |
| 65 | | X | X | | X | | | | | |
| 66 | | | | | | | | | | |
| 67 | | | | | | | | | | |
| 68 | X | X | X | X | X | | | | | |
| 69 | | | | | | | | | | |
| 70 | X | | | X | | | | | | |
| 71 | X | X | X | X | X | | | | | |
| 72 | X | | | | | | | | | |
| 73 | X | X | | X | X | | | | | |
| 74 | X | X | X | X | X | | | | | |
| 75 | X | X | X | X | X | | | | | |
| 76 | X | X | | X | X | | | | | |
| 77 | X | X | X | X | X | | | | | |
| 78 | X | X | X | X | X | | | | | |
| 79 | X | X | X | X | X | | | | | |
| 80 | X | X | X | X | X | | | | | |
| 81 | X | X | X | X | X | | | | | |
| 82 | X | X | X | X | X | | | | | |
| 83 | X | X | X | X | X | | | | | |
| 84 | X | X | X | X | X | | | | | |
| 85 | X | X | X | X | X | | | | | |
| 86 | X | X | X | X | X | | | | | |
| 87 | X | X | X | X | X | | | | | |
| 88 | X | X | X | X | X | | | | | |
| 89 | X | X | X | X | X | X | X | X | X | |
| 90 | X | X | X | X | X | X | X | X | X | |
| 91 | X | X | X | X | X | X | X | X | X | |
| 92 | X | X | X | X | X | X | X | X | X | |
| 93 | X | X | X | X | X | X | X | X | X | |
| 94 | | X | X | X | X | X | X | X | X | |
| 95 | X | X | X | X | X | X | X | X | X | |
| 96 | | X | X | X | X | X | X | X | X | |
| 97 | | X | X | X | X | X | X | X | X | |
| 98 | | X | X | X | X | X | X | X | X | |
| 99 | X | X | X | X | X | X | X | X | X | |
| 100 | X | X | X | X | X | X | X | X | X | |
| 101 | X | X | X | X | X | X | X | X | X | |
| 102 | X | X | X | X | X | X | X | X | X | |
| 103 | X | X | X | X | X | X | X | X | X | |
| 104 | X | X | X | X | X | X | X | X | X | |
| 105 | X | X | X | X | X | X | X | X | X | |
| 106 | X | X | X | X | X | X | X | X | X | |
| 107 | X | X | X | X | X | X | X | X | X | |
| 108 | X | X | X | X | X | X | X | X | X | |
| 109 | X | X | X | X | X | X | X | X | X | |
| 110 | X | X | X | X | X | X | X | X | X | |
| 111 | | X | X | X | X | X | X | X | X | |
| 112 | X | X | X | X | X | X | X | X | X | |
| 113 | X | | X | | X | X | X | X | X | |
| 114 | X | | X | | X | X | X | X | X | |
| 115 | X | | X | | X | X | X | X | X | |
| 116 | X | | X | | X | X | X | X | X | |
| 117 | X | | X | | X | X | X | X | X | |
| 118 | X | | X | | X | X | X | X | X | |
| 119 | X | | X | | X | X | X | X | X | |
| 120 | X | | X | | X | X | X | X | X | |
| 121 | X | X | X | X | X | X | X | X | X | |
| 122 | X | X | X | X | X | X | X | X | X | |
| 123 | X | X | X | X | X | X | X | X | X | |
| 124 | | X | X | X | X | X | X | X | X | |
| 125 | | X | X | X | X | X | X | X | | |
| 126 | X | X | X | X | X | X | X | X | | |
| 127 | X | X | X | X | X | X | X | X | | |
| 128 | X | X | X | X | X | X | X | X | | |
| 129 | X | X | X | X | X | X | X | X | | |
| 130 | | X | X | X | X | X | X | X | | |
| 131 | X | X | X | X | X | X | X | X | | |
| 132 | X | X | X | X | X | X | X | X | | |
| 133 | X | X | X | X | X | X | X | X | X | |
| 134 | X | X | X | X | X | X | X | X | X | |
| 135 | X | X | X | X | X | X | X | X | X | |
| 136 | X | X | X | X | X | X | X | X | X | |
| 137 | X | X | X | X | X | X | X | X | X | |
| 138 | X | X | X | X | X | X | X | X | X | |
| 139 | X | X | X | X | X | X | X | X | X | |
| 140 | X | X | X | X | X | X | X | X | X | |
| 141 | X | X | X | X | X | X | X | X | X | |
| 142 | X | X | X | X | X | X | X | X | X | |
| 143 | X | X | X | X | X | X | X | X | X | |
| 144 | X | X | X | X | X | X | X | X | X | |
| 145 | X | X | X | X | X | X | X | X | X | |
| 146 | X | X | X | X | X | X | X | X | X | |
| 147 | X | X | X | X | X | X | X | X | X | |
| 148 | X | X | X | X | X | X | X | X | X | |
| 149 | X | X | X | X | X | X | X | X | X | |
| 150 | X | X | X | X | X | X | X | X | X | |
| 151 | X | X | X | X | X | X | X | X | X | |
| 152 | X | X | X | X | X | X | X | X | X | |
| 153 | X | X | X | X | X | X | X | X | X | |
| 154 | X | X | X | X | X | X | X | X | X | |
| 155 | X | X | X | X | X | X | X | X | X | |
| 156 | X | X | X | X | X | X | X | X | X | |
| 157 | X | X | X | X | X | X | X | X | X | |
| 158 | X | X | X | X | X | X | X | X | X | |
| 159 | X | X | X | X | X | X | X | X | X | |
| 160 | X | X | X | X | X | X | X | X | X | |
| 161 | X | X | X | X | X | X | X | X | X | |
| 162 | X | X | X | X | X | X | X | X | X | |
| 163 | X | X | X | X | X | X | X | X | X | |
| 164 | X | X | X | X | X | X | X | X | X | |
| 165 | X | X | X | X | X | X | X | X | X | |
| 166 | X | X | X | X | X | X | X | X | X | |
| 167 | X | X | X | X | X | X | X | X | X | |
| 168 | X | X | X | X | X | X | X | X | X | |
| 169 | X | X | X | X | X | X | X | X | X | |
| 170 | X | X | X | X | X | X | X | X | X | |
| 171 | X | X | X | X | X | X | X | X | X | |
| 172 | X | X | X | X | X | X | X | X | X | |
| 173 | X | X | X | X | X | X | X | X | X | |
| 174 | X | X | X | X | X | X | X | X | X | |
| 175 | X | X | X | X | X | X | X | X | X | |
| 176 | X | X | X | X | X | X | X | X | X | |
| 177 | | | | X | X | X | | | X | |
| 178 | | | | X | X | X | | X | X | |
| 179 | | | X | X | X | X | | X | X | X |
| 180 | | | | | | | X | | | |
| 181 | | | X | X | X | X | X | X | | X |
| 182 | | | X | X | X | X | X | X | X | |
| 183 | | | | X | | X | | X | X | |
| 184 | | | | | | X | X | | | X |
| 185 | | | | | | X | | | | |
| 186 | | | | | | X | | | | |
| 187 | | | X | X | X | X | | | | |
| 188 | | | X | | | X | | X | | |
| 189 | | | X | X | X | X | X | X | X | |
| 190 | | | X | | | X | | | | |
| 191 | | | X | X | X | X | X | X | X | |
| 192 | | | | | | X | X | | | |
| 193 | | | X | | | X | | X | | |
| 194 | | | X | | | X | X | X | X | |
| 195 | | | | | | X | | | | X |
| 196 | | | X | X | | X | X | X | X | |
| 197 | | | X | X | X | X | X | X | X | |
| 198 | | X | X | X | | X | X | X | X | |
| 199 | | X | X | X | X | X | X | X | X | |
| 200 | | X | | X | | | X | | | |
| 201 | | X | X | | X | X | X | X | X | |
| 202 | | | X | | | X | X | | | |
| 203 | | X | X | | | | X | X | | |
| 204 | | | X | | | X | X | | | |
| 205 | | X | | | X | X | | X | X | |
| 206 | | X | X | X | X | X | X | X | X | X |
| 207 | | | | X | | X | | | | |
| 208 | | X | X | X | X | X | | X | X | |
| 209 | | | | | | X | X | X | | |
| 210 | | | X | X | X | X | X | | X | |
| 211 | | X | X | | | X | X | | | |
| 212 | | | | | X | X | X | | | |
| 213 | | | | | X | | X | | | |
| 214 | | | | X | | X | | | | |
| 215 | | | | | | | | | | |
| 216 | X | X | | X | X | X | X | X | X | |
| 217 | X | X | X | | X | X | X | X | X | |
| 218 | X | X | X | X | | X | X | X | X | |
| 219 | X | X | X | X | | X | | X | X | |
| 220 | X | X | X | X | | X | | X | X | |
| 221 | X | X | X | X | | X | X | X | X | |
| 222 | | | | | | X | | X | X | |
| 223 | | X | | | | | | | X | |
| 224 | X | X | | | X | | X | X | X | |
| 225 | | X | | | | | | | | |
| 226 | X | X | X | | X | | | X | X | |
| 227 | X | | | | | X | X | X | X | |
| 228 | X | X | | | X | X | X | X | X | X |
| 229 | | | | | | | | | X | |
| 230 | X | X | X | | | X | X | X | X | |
| 231 | X | X | X | X | X | X | X | X | X | |
| 232 | X | X | | | | | X | X | X | |
| 233 | X | X | | | | | X | X | X | |
| 234 | | X | | | X | | X | | | |
| 235 | X | X | X | X | | X | X | X | X | |
| 236 | X | X | X | X | | X | | X | X | |
| 237 | X | X | X | X | X | X | X | X | X | |
| 238 | X | X | | X | X | X | X | X | X | |
| 239 | X | X | X | X | X | X | X | X | X | |
| 240 | X | X | X | X | X | X | X | X | X | |
| 241 | X | X | X | X | X | X | X | X | X | |
| 242 | X | X | X | X | X | X | X | X | X | |
| 243 | X | X | X | X | X | X | X | X | X | |
| 244 | X | X | X | X | X | X | | X | | |
| 245 | X | X | X | X | X | X | X | X | X | |
| 246 | X | X | | X | | X | X | X | X | |
| 247 | X | X | X | X | X | X | X | X | X | |
| 248 | X | X | X | X | X | X | X | X | X | |
| 249 | X | X | X | X | X | X | X | X | X | |
| 250 | X | X | X | X | X | X | X | X | X | |
| 251 | X | X | X | X | X | X | X | X | X | |
| 252 | X | X | X | X | X | X | X | X | X | |
| 253 | X | X | X | X | X | X | | X | X | |
| 254 | X | X | X | X | X | X | X | X | X | |
| 255 | X | X | X | X | X | X | X | X | X | |
| 256 | X | X | X | X | X | X | X | X | X | |
| 257 | X | X | X | X | X | X | X | X | X | |
| 258 | X | X | X | X | X | X | X | X | X | |
| 259 | X | X | X | X | X | X | X | X | X | |
| 260 | X | X | X | X | X | X | X | X | X | |
| 261 | X | X | X | X | X | X | X | X | X | |
| 262 | X | | X | X | X | X | | | X | |
| 263 | X | X | X | X | X | X | X | X | X | |
| 264 | X | X | X | X | X | X | X | X | X | |
| 265 | X | | X | X | X | X | X | X | X | |
| 266 | X | | X | X | X | X | X | X | X | |
| 267 | X | | X | X | X | X | X | X | X | |
| 268 | X | X | X | X | X | X | X | X | X | |
| 269 | X | X | X | X | | X | X | X | X | |
| 270 | X | X | | X | | X | X | X | X | |
| 271 | X | X | X | X | X | X | X | X | | |
| 272 | X | | X | X | X | X | | X | X | |
| 273 | X | X | X | X | X | X | X | X | X | |
| 274 | X | | X | X | X | X | | X | X | |
| 275 | X | X | X | X | X | X | X | X | X | |
| 276 | X | X | X | X | X | X | X | X | X | |
| 277 | X | | X | X | X | X | X | X | X | |
| 278 | X | | | X | | X | X | | | |

TABLE E
R¹ Groups

| | |
|---|---|
| | 1 |
| 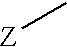 | |
| | 2 |
| 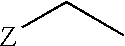 | |
| | 3 |
| 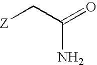 | |
| | 4 |
| 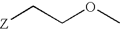 | |
| | 5 |
| 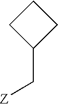 | |
| | 6 |
| 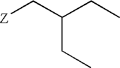 | |
| | 7 |
| 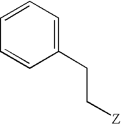 | |
| | 8 |
| 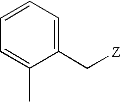 | |
| | 9 |
| 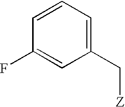 | |
| | 10 |
| 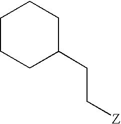 | |
| | 11 |
| 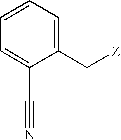 | |
| | 12 |
| 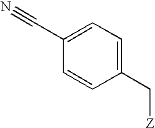 | |
| | 13 |
| 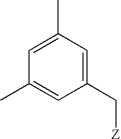 | |
| | 14 |
| 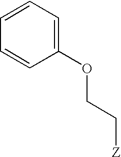 | |
| | 15 |
| 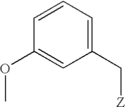 | |
| | 16 |
| 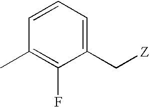 | |
| | 17 |
| 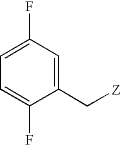 | |
| | 18 |
| 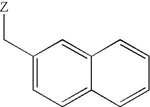 | |
| | 19 |
| 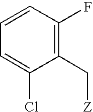 | |
| | 20 |
| 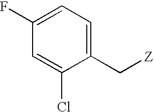 | |
| | 21 |
| 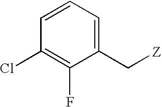 | |
| | 22 |
| 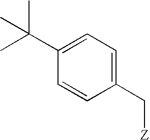 | |
| | 23 |
| 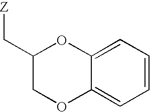 | |
| | 24 |
| 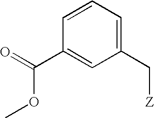 | |
| | 25 |
| 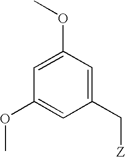 | |
| | 26 |
| 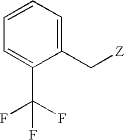 | |
| | 27 |
| 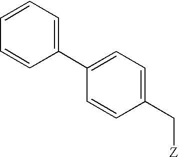 | |
| | 28 |
| 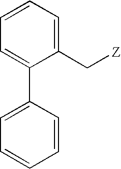 | |
| | 29 |
| 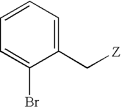 | |
| | 30 |
| 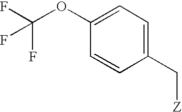 | |
| | 31 |
| 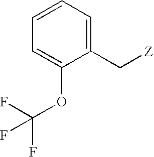 | |
| | 32 |
| 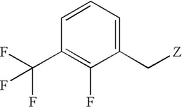 | |
| | 33 |
| 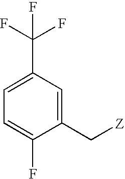 | |
| | 34 |
| 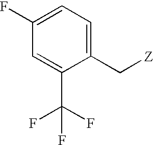 | |
| | 35 |
| 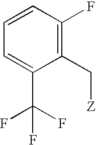 | |
| | 36 |
| 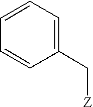 | |
| | 37 |
| 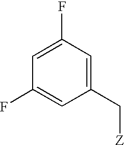 | |
| | 38 |
| 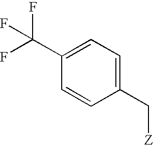 | |
| | 39 |
| 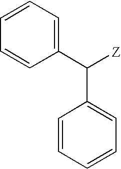 | |
| | 40 |
| 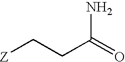 | |

wherein Z is the point of attachment of group R¹ to the compounds of formula (I).

TABLE F
R² Groups

| | |
|---|---|
| | 1 |
| 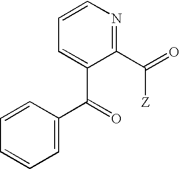 | |
| | 2 |
| 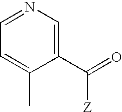 | |
| | 3 |
| 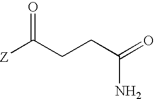 | |
| | 4 |
| 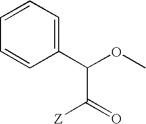 | |
| | 5 |
| 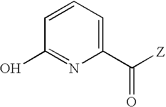 | |
| | 6 |
| 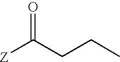 | |
| | 7 |
| 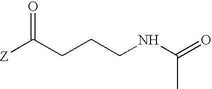 | |
| | 8 |
| 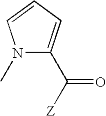 | |
| | 9 |
| 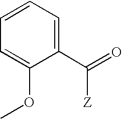 | |
| | 10 |
| 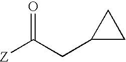 | |
| | 11 |
| 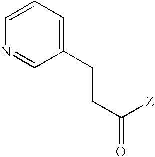 | |
| | 12 |
| 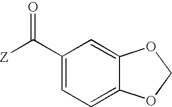 | |
| | 13 |
| 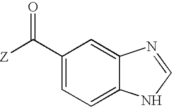 | |
| | 14 |
| 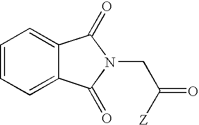 | |
| | 15 |
| 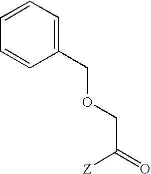 | |
| | 16 |
| 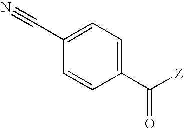 | |
| | 17 |
| 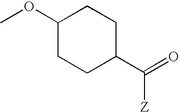 | |
| | 18 |
| 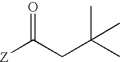 | |
| | 19 |
| 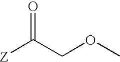 | |
| | 20 |
| 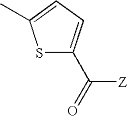 | |
| | 21 |
| 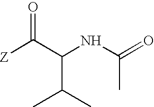 | |
| | 22 |
| 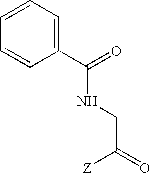 | |
| | 23 |
| 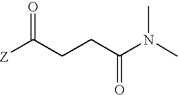 | |
| | 24 |
| 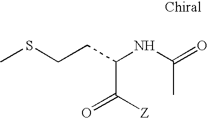 | |
| | 25 |
| 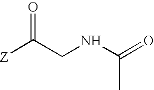 | |
| | 26 |
| 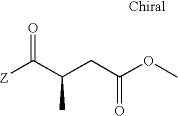 | |
| | 27 |
| 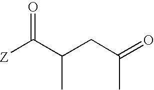 | |
| | 28 |
| 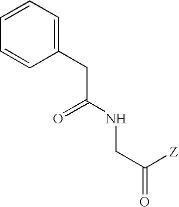 | |
| | 29 |
| 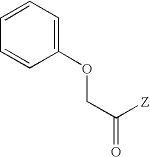 | |
| | 30 |
| 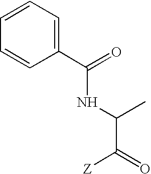 | |
| | 31 |
| 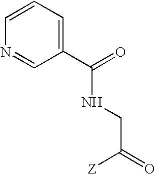 | |
| | 32 |
| 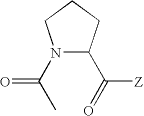 | |
| | 33 |
| 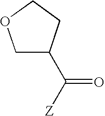 | |
| | 34 |
| 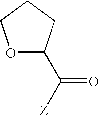 | |
| | 35 |
| 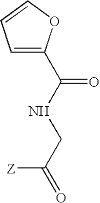 | |
| | 36 |
| 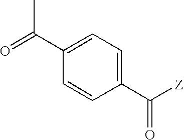 | |
| | 37 |
| 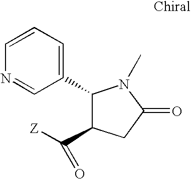 | |
| | 38 |
| 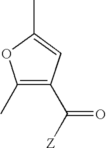 | |
| | 39 |
| 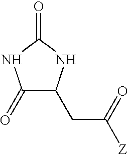 | |
| | 40 |
| 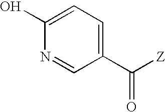 | |
| | 41 |
| 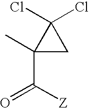 | |
| | 42 |
| 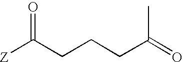 | |
| | 43 |
| 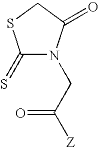 | |
| | 44 |
| 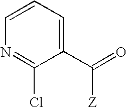 | |
| | 45 |
| 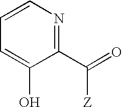 | |
| | 46 |
| 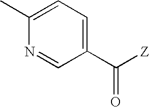 | |
| | 47 |
| 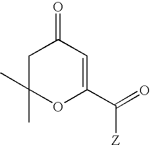 | |
| | 48 |
| 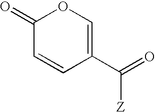 | |
| | 49 |
| 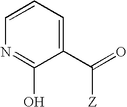 | |
| | 50 |
| 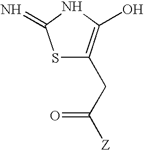 | |
| | 51 |
| 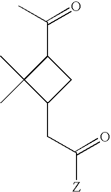 | |
| | 52 |
| 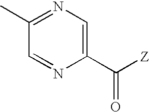 | |
| | 53 |
| 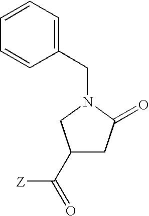 | |
| | 54 |
| 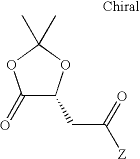 | |
| | 55 |
| 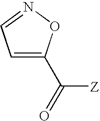 | |
| | 56 |
| 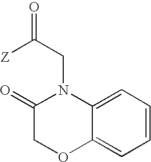 | |
| | 57 |
| 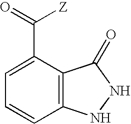 | |
| | 58 |
| 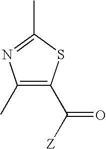 | |
| | 59 |
| 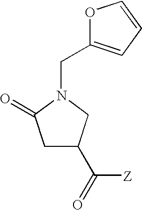 | |
| | 60 |
| 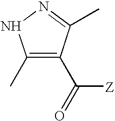 | |
| | 61 |
| 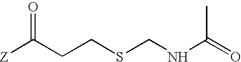 | |
| | 62 |
| 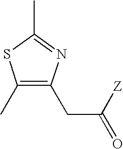 | |
| | 63 |
| 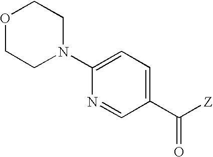 | |
| | 64 |
| 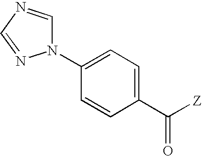 | |
| | 65 |
| 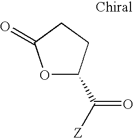 | |
| | 66 |
| 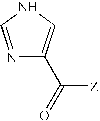 | |
| | 67 |
| 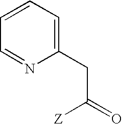 | |
| | 68 |
| 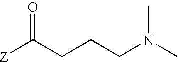 | |
| | 69 |
| 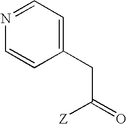 | |
| | 70 |
| 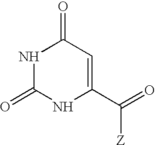 | |
| | 71 |
| 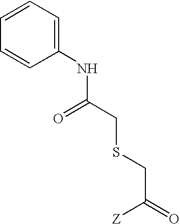 | |
| | 72 |
| 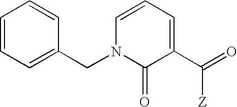 | |
| | 73 |
| 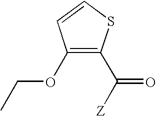 | |
| | 74 |
| 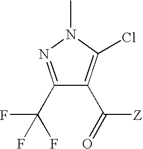 | |
| | 75 |
| 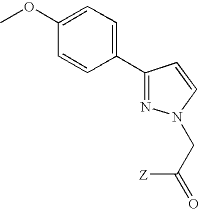 | |
| | 76 |
| 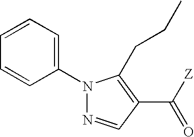 | |
| | 77 |
| 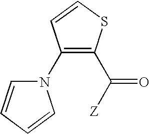 | |
| | 78 |
| 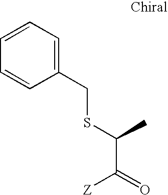 | |
| | 79 |
| 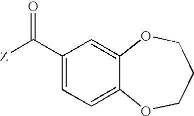 | |
| | 80 |
| 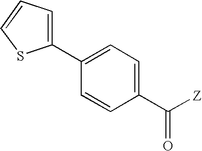 | |
| | 81 |
| 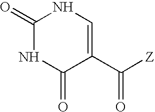 | |
| | 82 |
| 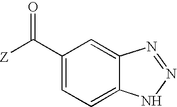 | |
| | 83 |
| 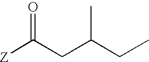 | |
| | 84 |
| 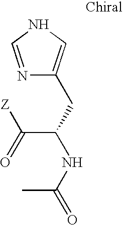 | |
| | 85 |
| 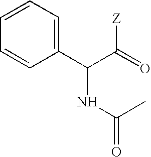 | |
| | 86 |
| 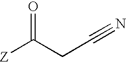 | |
| | 87 |
| 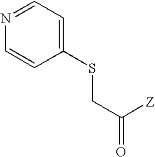 | |
| | 88 |
| 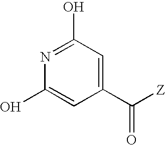 | |
| | 89 |
| 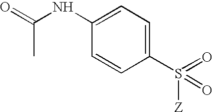 | |
| | 90 |
| 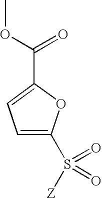 | |
| | 91 |
| 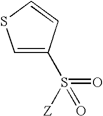 | |
| | 92 |
| 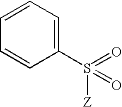 | |
| | 93 |
| 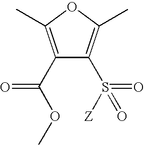 | |
| | 94 |
| 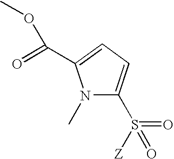 | |
| | 95 |
| 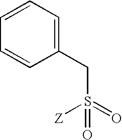 | |
| | 96 |
| 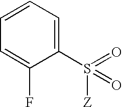 | |
| | 97 |
| 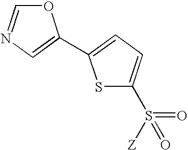 | |
| | 98 |
| 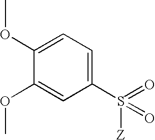 | |
| | 99 |
| 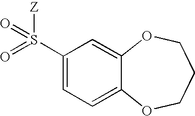 | |

| | |
|---|---|
| | 100 |
| 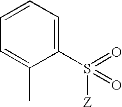 | |
| | 101 |
| 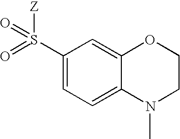 | |
| | 102 |
| 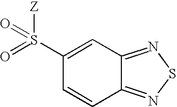 | |
| | 103 |
| 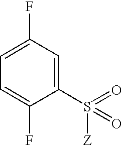 | |
| | 104 |
| 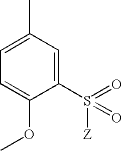 | |
| | 105 |
| 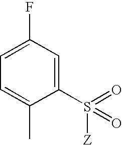 | |
| | 106 |
| 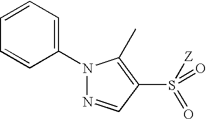 | |
| | 107 |
| 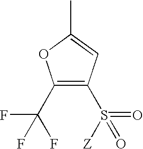 | |
| | 108 |
| 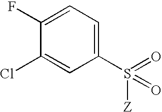 | |
| | 109 |
| 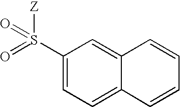 | |
| | 110 |
| 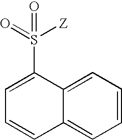 | |
| | 111 |
| 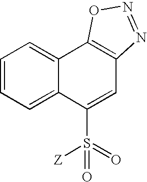 | |
| | 112 |
| 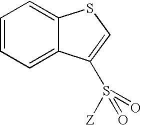 | |
| | 113 |
| 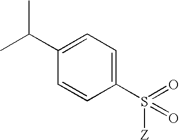 | |
| | 114 |
| 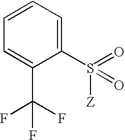 | |
| | 115 |
| 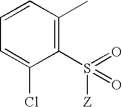 | |
| | 116 |
| 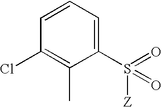 | |
| | 117 |
| 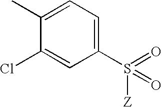 | |
| | 118 |
| 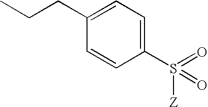 | |
| | 119 |
| 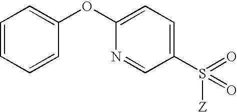 | |
| | 120 |
| 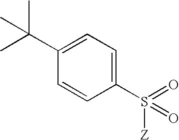 | |
| | 121 |
| 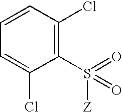 | |
| | 122 |
| 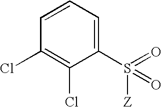 | |
| | 123 |
| 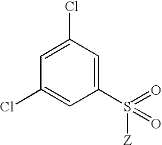 | |
| | 124 |
| 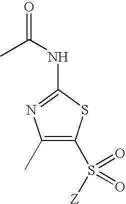 | |
| | 125 |
| 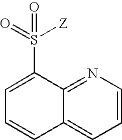 | |
| | 126 |
| 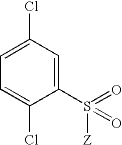 | |
| | 127 |
| 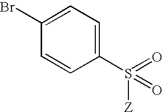 | |
| | 128 |
| 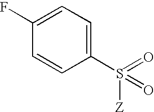 | |
| | 129 |
| 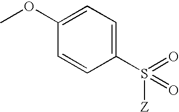 | |
| | 130 |
| 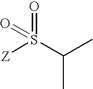 | |
| | 131 |
| 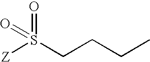 | |
| | 132 |
| 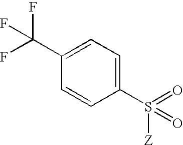 | |
| | 133 |
| 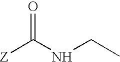 | |
| | 134 |
| 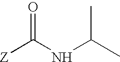 | |
| | 135 |
| 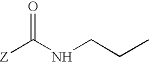 | |
| | 136 |
| 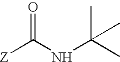 | |
| | 137 |
| 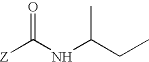 | |
| | 138 |
| 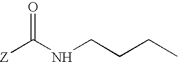 | |
| | 139 |
| 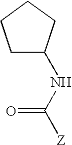 | |
| | 140 |
| 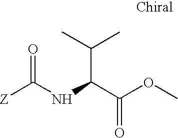 | |
| | 141 |
| 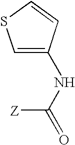 | |
| | 142 |
| 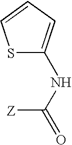 | |
| | 143 |
| 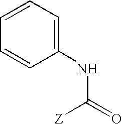 | |
| | 144 |
| 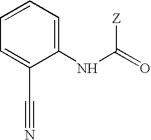 | |
| | 145 |
| 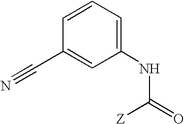 | |
| | 146 |
| 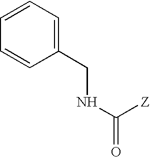 | |
| | 147 |
| 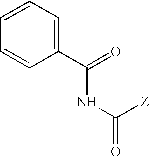 | |
| | 148 |
| 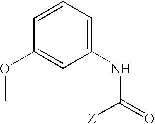 | |
| | 149 |
| 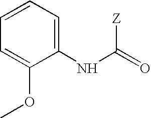 | |
| | 150 |
| 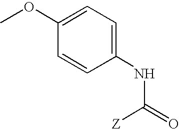 | |
| | 151 |
| 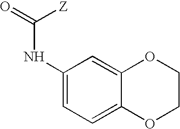 | |
| | 152 |
| 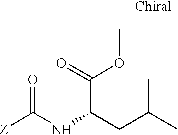 | |
| | 153 |
| 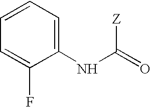 | |
| | 154 |
| 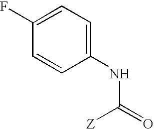 | |
| | 155 |
| 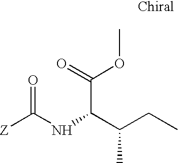 | |
| | 156 |
| 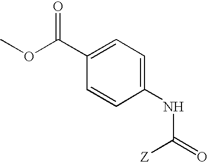 | |
| | 157 |
| 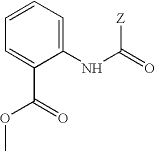 | |
| | 158 |
| 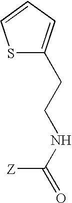 | |
| | 159 |
| 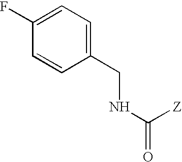 | |
| | 160 |
| 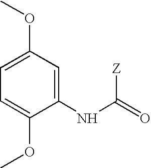 | |
| | 161 |
| 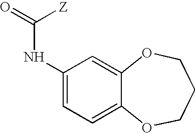 | |
| | 162 |
| 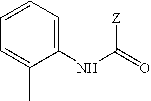 | |
| | 163 |
| 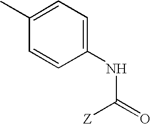 | |
| | 164 |
| 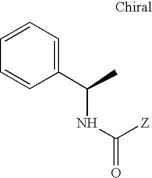 | |
| | 165 |
| 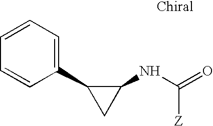 | |
| | 166 |
| 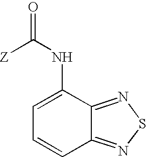 | |
| | 167 |
| 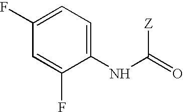 | |
| | 168 |
| 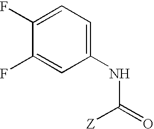 | |
| | 169 |
| 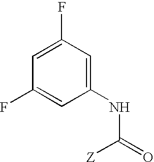 | |
| | 170 |
| 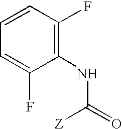 | |
| | 171 |
| 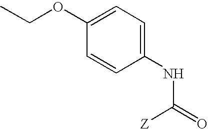 | |
| | 172 |
| 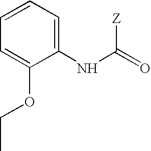 | |
| | 173 |
| 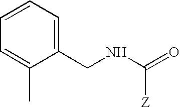 | |
| | 174 |
| 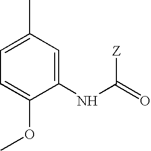 | |
| | 175 |
| 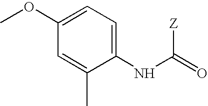 | |
| | 176 |
| 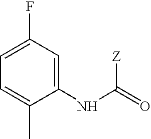 | |
| | 177 |
| 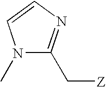 | |
| | 178 |
| 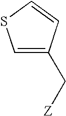 | |
| | 179 |
| 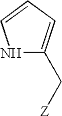 | |
| | 180 |
| 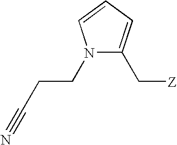 | |
| | 181 |
| 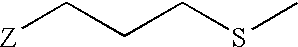 | |
| | 182 |
| 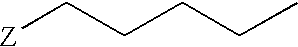 | |
| | 183 |
| 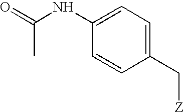 | |
| | 184 |
| 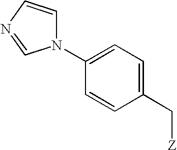 | |
| | 185 |
| 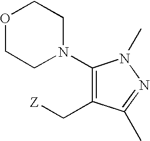 | |
| | 186 |
| 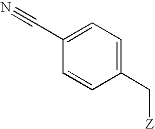 | |
| | 187 |
| 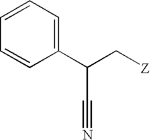 | |
| | 188 |
| 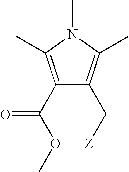 | |
| | 189 |
| 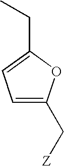 | |
| | 190 |
| 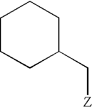 | |
| | 191 |
| 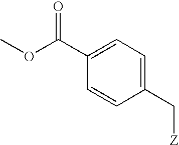 | |
| | 192 |
| 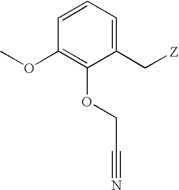 | |
| | 193 |
| 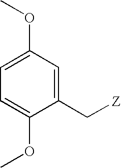 | |
| | 194 |
| 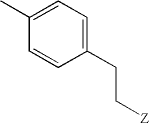 | |
| | 195 |
| 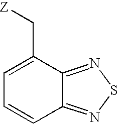 | |
| | 196 |
| 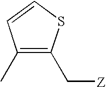 | |
| | 197 |
| 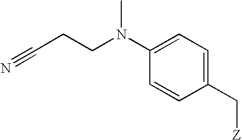 | |
| | 198 |
| 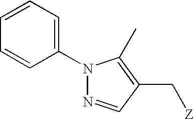 | |
| | 199 |
| 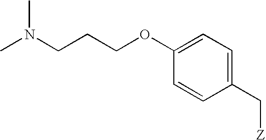 | |
| | 200 |
| 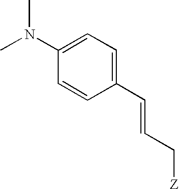 | |
| | 201 |
| 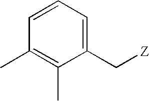 | |
| | 202 |
| 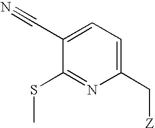 | |
| | 203 |
| 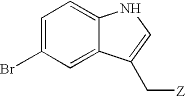 | |
| | 204 |
| 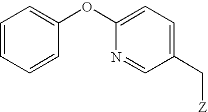 | |
| | 205 |
| 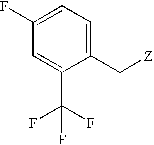 | |
| | 206 |
| 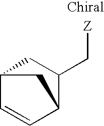 | |
| | 207 |
| 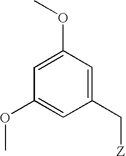 | |
| | 208 |
| 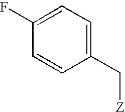 | |
| | 209 |
| 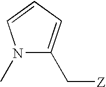 | |
| | 210 |
| 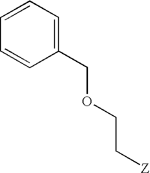 | |
| | 211 |
| 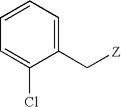 | |
| | 212 |
| 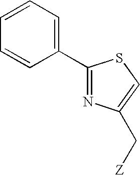 | |
| | 213 |
| 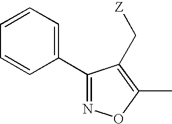 | |
| | 214 |
| 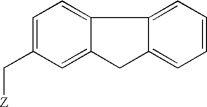 | |
| | 215 |
| 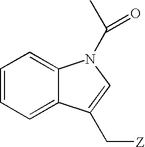 | |
| | 216 |
| 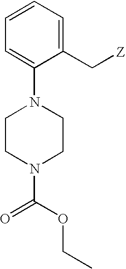 | |
| | 217 |
| 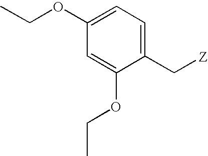 | |
| | 218 |
| 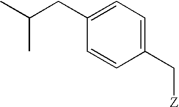 | |
| | 219 |
| 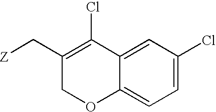 | |
| | 220 |
| 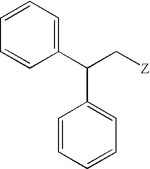 | |
| | 221 |
| 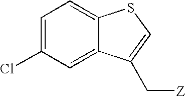 | |
| | 222 |
| 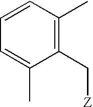 | |
| | 223 |
| 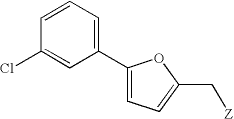 | |
| | 224 |
| 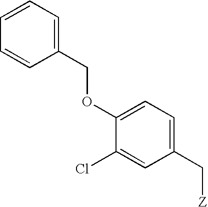 | |
| | 225 |
| 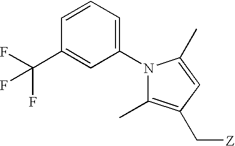 | |
| | 226 |
| 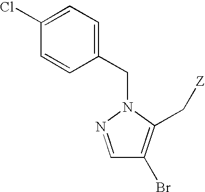 | |
| | 227 |
| 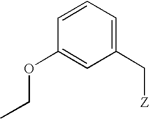 | |
| | 228 |
| 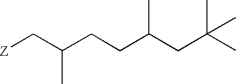 | |
| | 229 |
| 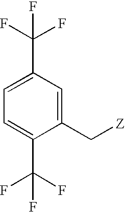 | |
| | 230 |
| 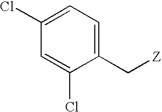 | |
| | 231 |
| 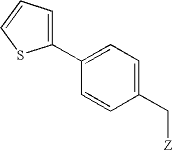 | |
| | 232 |
| 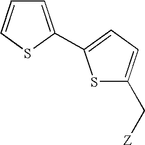 | |
| | 233 |
| 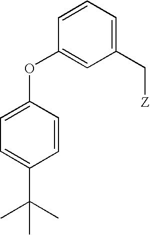 | |
| | 234 |
| 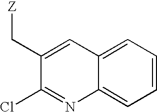 | |
| | 235 |
| 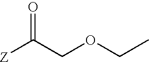 | |
| | 236 |
| 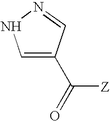 | |
| | 237 |
| 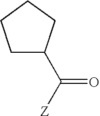 | |
| | 238 |
| 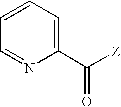 | |
| | 239 |
| 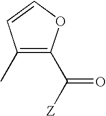 | |
| | 240 |
| 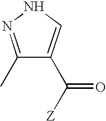 | |
| | 241 |
| 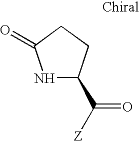 | |
| | 242 |
| 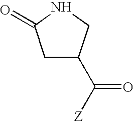 | |
| | 243 |
| 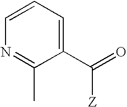 | |
| | 244 |
| 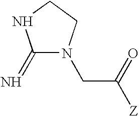 | |
| | 245 |
| 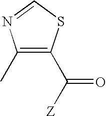 | |
| | 246 |
| 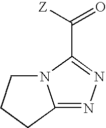 | |
| | 247 |
| 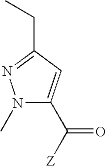 | |
| | 248 |
| 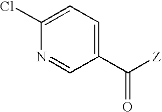 | |
| | 249 |
| 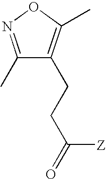 | |
| | 250 |
| 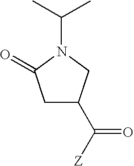 | |
| | 251 |
| 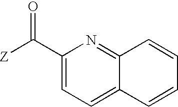 | |
| | 252 |
| 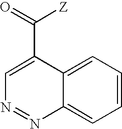 | |
| | 253 |
| 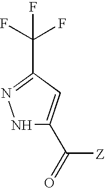 | |
| | 254 |
| 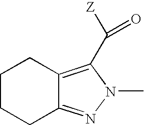 | |
| | 255 |
| 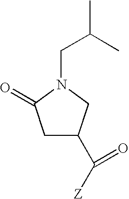 | |
| | 256 |
| 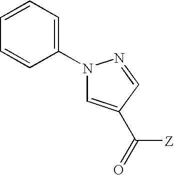 | |
| | 257 |
| 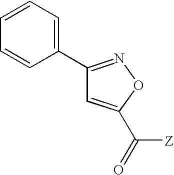 | |
| | 258 |
| 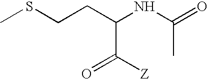 | |
| | 259 |
| 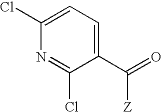 | |
| | 260 |
| 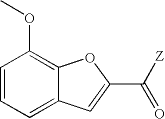 | |
| | 261 |
| 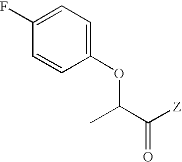 | |
| | 262 |
| 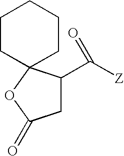 | |
| | 263 |
| 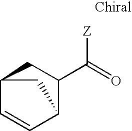 | |
| | 264 |
| 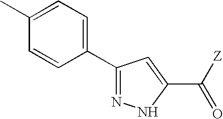 | |
| | 265 |
| 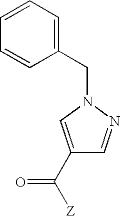 | |
| | 266 |
| 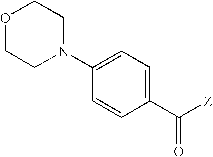 | |
| | 267 |
| 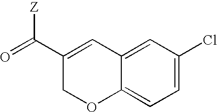 | |
| | 268 |
| 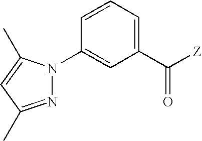 | |
| | 269 |
| 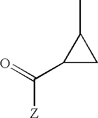 | |
| | 270 |
| 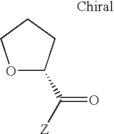 | |
| | 271 |
| 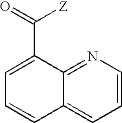 | |
| | 272 |
| 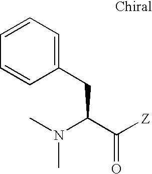 | |
| | 273 |
| 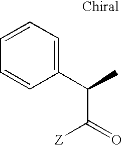 | |
| | 274 |
| 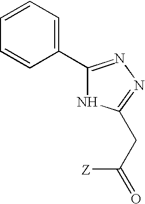 | |
| | 275 |
| 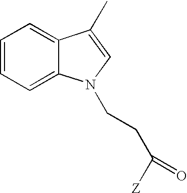 | |
| | 276 |
| 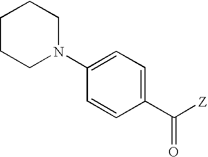 | |
| | 277 |
| 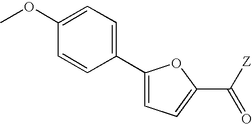 | |
| | 278 |
| 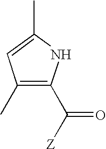 | |

wherein Z is the point of attachment of group R² to the compounds of formula (I).

The compounds described by formula (I) and defined by an “X” in Tables A-D have the R¹ and R² definitions as indicated by an “X” in the box formed by the intersection of the R¹ column and the R² row, and are within the scope of the present invention. The numbers in the top row of Tables A-D represent the R¹ groups defined in Table E. The numbers in the leftmost column in Tables A-D represent the R² groups defined in Table F. The compounds represented by blank boxes in Tables A-D are excluded from the scope of the present invention.

Any occurrence of the word “chiral” in Table F refers to the R² group situated directly below the word “chiral.”

The compounds of formula (I) or pharmaceutically acceptable salts, solvates, esters or prodrugs thereof (referred to herein as the “Tetrahydropyrido[4,3-d]Pyrimidinone Derivatives”) can be useful for treating or preventing obesity, diabetes, metabolic syndrome, a cardiovascular disease or a disorder related to the activity of GPR119 (each being a “Condition”) in a patient.

The details of the invention are set forth in the accompanying detailed description below.

Although any methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

In an embodiment, the present invention provides methods of using the Tetrahydropyrido[4,3-d]Pyrimidinone Derivatives for treating or preventing a Condition in a patient.

### Definitions and Abbreviations

As used above, and throughout this disclosure, the following terms, unless otherwise indicated, shall be understood to have the following meanings:

As used above, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “obesity” as used herein, refers to a patient being overweight and having a body mass index (BMI) of 25 or greater. In one embodiment, an obese patient has a BMI of about 25 or greater. In another embodiment, an obese patient has a BMI of between about 25 and about 30. In another embodiment, an obese patient has a BMI of between about 35 and about 40. In still another embodiment, an obese patient has a BMI greater than 40.

The term “obesity-related disorder” as used herein refers to: (i) disorders which result from a patient having a BMI of about 25 or greater; and (ii) eating disorders and other disorders associated with excessive food intake. Non-limiting examples of an obesity-related disorder include edema, shortness of breath, sleep apnea, skin disorders and high blood pressure.

The term “metabolic syndrome” as used herein, refers to a set of risk factors that make a patient more succeptible to cardiovascular disease and/or type 2 diabetes. As defined herein, a patient is considered to have metabolic syndrome if the patient has one or more of the following five risk factors:
1) central/abdominal obesity as measured by a waist circumference of greater than 40 inches in a male and greater than 35 inches in a female;
2) a fasting triglyceride level of greater than or equal to 150 mg/dL;
3) an HDL cholesterol level in a male of less than 40 mg/dL or in a female of less than 50 mg/dL;
4) blood pressure greater than or equal to 130/85 mm Hg; and
5) a fasting glucose level of greater than or equal to 110 mg/dL.

The term “effective amount” as used herein, refers to an amount of compound of formula (I) and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

The term “purified”, “in purified form” or “in isolated and purified form” for a compound refers to the physical state of the compound after being isolated from a synthetic process (e.g. from a reaction mixture), or natural source or combination thereof. Thus, the term “purified”, “in purified form” or “in isolated and purified form” for a compound refers to the physical state of the compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a Tetrahydropyrido[4,3-d]Pyrimidinone Derivative or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a Tetrahydropyrido[4,3-d]Pyrimidinone Derivative or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Tetrahydropyrido[4,3-d]Pyrimidinone Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C_{I}-C₆)alkoxycarbonyloxymethyl, N—(C₁-C₆)alkoxycarbonylaminomethyl, succinoyl, (C_{I}-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a Tetrahydropyrido[4,3-d]Pyrimidinone Derivative incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, C(Y⁴)Y⁵ wherein Y⁴ is H or methyl and Y⁵ is mono-N or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. “Hydrate” is a solvate wherein the solvent molecule is H₂O.

One or more compounds of the invention may optionally be converted to a solvate. Preparation of Solvates is Generally Known. Thus, for Example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The Tetrahydropyrido[4,3-d]Pyrimidinone Derivatives can form salts which are also within the scope of this invention. Reference to a Tetrahydropyrido[4,3-d]Pyrimidinone Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Tetrahydropyrido[4,3-d]Pyrimidinone Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a Tetrahydropyrido[4,3-d]Pyrimidinone Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Tetrahydropyrido[4,3-d]Pyrimidinone Derivatives may be atropisomers (e.g., substituted biaryls) and are considered as part of this invention. Enantiomers can also be separated by use of chiral HPLC column.

It is also possible that the Tetrahydropyrido[4,3-d]Pyrimidinone Derivatives may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a Tetrahydropyrido[4,3-d]Pyrimidinone Derivative incorporates a double bond or a fused ring, both the cis- and trans-forms, as well as mixtures, are embraced within the scope of the invention. Also, for example, all keto-enol and imine-enamine forms of the compounds are included in the invention.).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Tetrahydropyrido[4,3-d]Pyrimidinone Derivatives (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled Tetrahydropyrido[4,3-d]Pyrimidinone Derivatives can generally be prepared using synthetic chemical procedures analogous to those disclosed herein for making the Compounds of Formula (I), by substituting an appropriate isotopically labelled starting material or reagent for a non-isotopically labelled starting material or reagent.

Polymorphic forms of the Tetrahydropyrido[4,3-d]Pyrimidinone Derivatives, and of the salts, solvates, hydrates, esters and prodrugs of the Tetrahydropyrido[4,3-d]Pyrimidinone Derivatives, are intended to be included in the present invention.

The following abbreviations are used below and have the following meanings: Boc or BOC is C(O)O-(t-butyl), BSA is bovine serum albumin, DCC is N,N-dicyclohexylcarbodiimide; DCE is dichloroethane, DMAP is 4-dimethylaminopyridine, DMEM is Dulbecco's modified eagle medium, DMF is N,N-dimethylformamide, EDC is 1-ethyl-3-(3-dimethylaminopropyl)-carbodiimide hydrochloride; EtOAc is ethyl acetate, EtOH is ethanol, Et₃N is triethylamine, HOAc is acetic acid, HOBt is N-hydroxybenzotriazole, MeCN is acetonitrile, MeOH is methanol and THF is tetrahydrofuran.

### The Tetrahydropyrido[4,3-d]Pyrimidinone Derivatives of Formula (I)

The present invention provides methods for treating or preventing a Condition in a patient, the methods comprising administering to the patient an effective amount of one or more compounds of Formula (I):

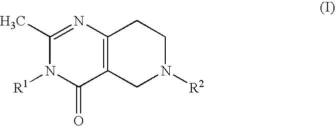

and pharmaceutically acceptable salts, solvates, esters and prodrugs thereof, wherein R¹ and R² are defined above for the compounds of formula (I).

In one embodiment, a compound of formula (I) is in purified form.

### Methods For Making the Compounds of Formula (I)

Methods useful for making the Tetrahydropyrido[4,3-d]Pyrimidinone Derivatives of Formula (I) are set forth in the Examples below and generalized in Schemes 1-6. Alternative synthetic pathways and analogous structures within the scope of the invention may be apparent to those skilled in the art.

Scheme 1 shows a method useful for making compound C, which is a useful intermediate for making the Tetrahydropyrido[4,3-d]Pyrimidinone Derivatives.

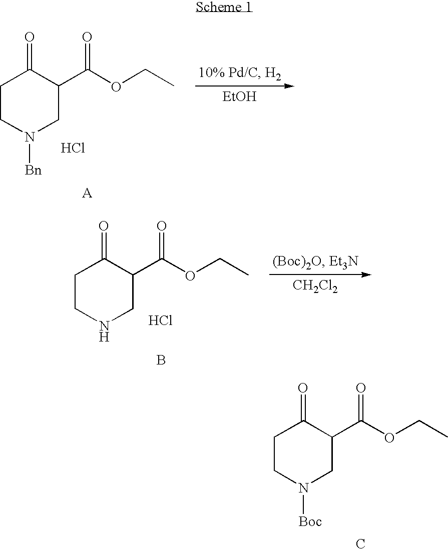

A 4-Oxo-N-benzyl piperidinyl compound of formula A can be deprotected via catalytic hydrogenation using Pd/C to provide the 4-Oxo-piperidinyl compound B. The cyclic amine group of compound B can then be reprotected as its N-tert-butyloxycarbonyl (BOC) derivative to provide intermediate compound C using BOC-anhydride and triethylamine.

Scheme 2 shows a method for making the intermediate piperidine hydrochloride compounds of formula H which are useful intermediates for making the Tetrahydropyrido[4,3-d]Pyrimidinone Derivatives.

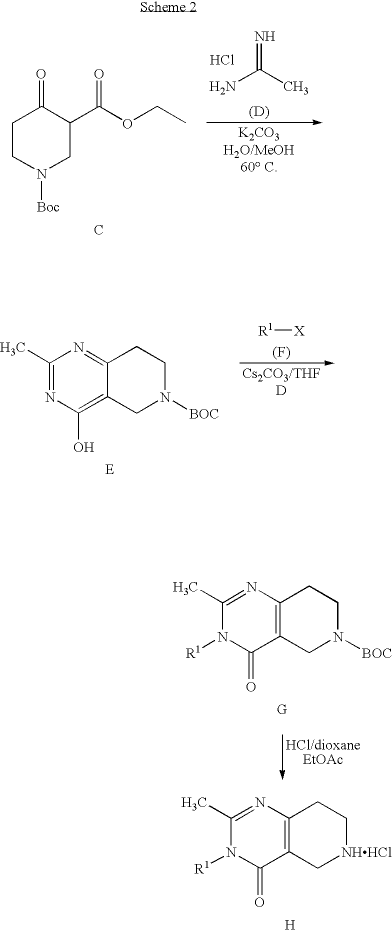

wherein R¹ is defined above for the compounds of formula (I) and X is a good leaving group, such as —Cl, —Br, —I, —O-tosyl, —O-mesyl or —O-triflyl.

Compound C can be reacted with an amidine hydrochloride compound of formula D to provide the pyrimidino-piperidine compounds of formula E, which can then be reacted with a compound of formula F in the presence of a carbonate base in THF or DMF to provide the substituted pyrimidinone compounds of formula G (NaI may be added to assist in the reaction of more slowly reactive compounds of formula F). The BOC protecting group of a compound of formula G can then be removed using HCl to provide the piperidine hydrochloride compounds of formula H.

Scheme 3 shows a method for converting intermediate compounds of formula H to the Tetrahydropyrido[4,3-d]Pyrimidinone Derivatives, wherein R² is joined via a methylene group.

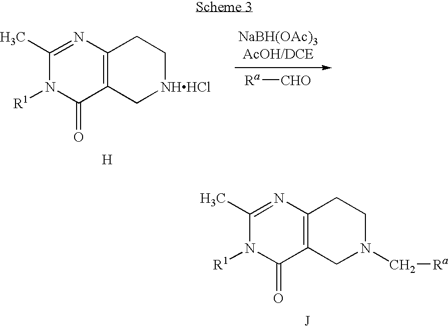

wherein R¹ is defined above for the compounds of formula (I) and CH₂R^{a} is representative of all R² substituents, as defined for the compounds of formula (I), that are connected via a methylene group.

The amine hydrochloride compounds of formula H can be reacted with an aldehyde of formula R^{a}—CHO, followed by reduction of the resulting imine using NaBH(OAc)₃ to provide the compounds of formula J, which correspond to the compounds of formula (I) wherein R² is a substituent that is connected via a methylene group.

Scheme 4 shows a method for converting intermediate compounds of formula H to the Tetrahydropyrido[4,3-d]Pyrimidinone Derivatives, wherein R² is joined via a SO₂— group.

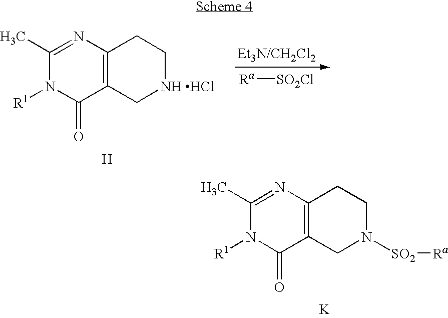

wherein R¹ is defined above for the compounds of formula (I) and —S(O)₂R^{a} is representative of all R² substituents, as defined for the compounds of formula (I), that are connected via a S(O)₂— group.

The amine hydrochloride compounds of formula H can be reacted with a compound of formula R^{a}—SO₂Cl in the presence of a non-nucleophilic base, such as Et₃N, to provide the compounds of formula K, which correspond to the compounds of formula (I) wherein R² is a substituent that is connected via a S(O)₂— group.

Scheme 5 shows a method for converting intermediate compounds of formula H to the Tetrahydropyrido[4,3-d]Pyrimidinone Derivatives, wherein R² is joined via a C(O)NH— group.

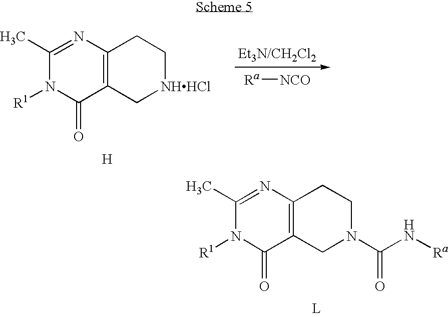

wherein R¹ is defined above for the compounds of formula (I) and —C(O)NHR^{a} is representative of all R² substituents, as defined for the compounds of formula (I), that are connected via a —C(O)NH— group.

The amine hydrochloride compounds of formula H can be reacted with an isocyanate of formula R^{a}—NCO, in the presence of a non-nucleophilic base, such as Et₃N, to provide the compounds of formula L, which correspond to the compounds of formula (I) wherein R² is a substituent that is connected via a C(O)NH— group.

Scheme 6 shows a method for converting intermediate compounds of formula H to the Tetrahydropyrido[4,3-d]Pyrimidinone Derivatives, wherein R² is joined via a C(O)— group.

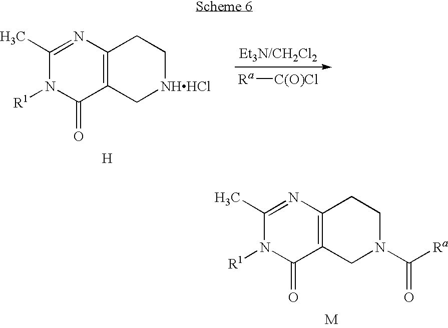

wherein R¹ is defined above for the compounds of formula (I) and C(O)R^{a} is representative of all R² substituents, as defined for the compounds of formula (I), that are connected via a C(O)— group.

The amine hydrochloride compounds of formula H can be reacted with an acid chloride of formula R^{a}—C(O)C1 or an appropriate mixed anhydride, in the presence of a non-nucleophilic base, such as Et₃N, to provide the compounds of formula M, which correspond to the compounds of formula (I) wherein R² is a substituent that is connected via a C(O)— group.

Scheme 7 shows an alternative method for converting intermediate compounds of formula H to the Tetrahydropyrido[4,3-d]Pyrimidinone Derivatives, wherein R² is joined via a C(O)— group.

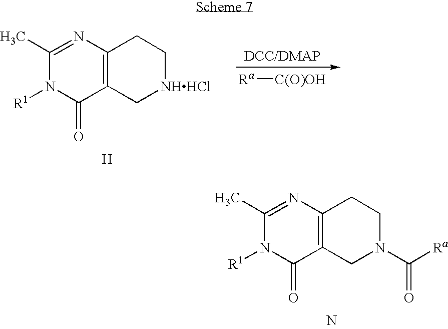

wherein R¹ is defined above for the compounds of formula (I) and C(O)R^{a} is representative of all R² substituents, as defined for the compounds of formula (I), that are connected via a C(O)— group.

The amine hydrochloride compounds of formula H can be coupled with an acid of formula R^{a}—C(O)OH in the presence of, for example DCC and DMAP, or any other suitable coupling agent, to provide the compounds of formula N, which correspond to the compounds of formula (I) wherein R² is a substituent that is connected via a C(O)— group.

It is to be understood that the amine hydrochloride compounds of formula H can be replaced with the corresponding free amine as starting materials in the methods set forth above in schemes 3-7.

The starting materials and reagents depicted in Schemes 1-6 are either available from commercial suppliers such as Sigma-Aldrich (St. Louis, Mo.) and Acros Organics Co. (Fair Lawn, N.J.), or can be prepared using methods well-known to those of skill in the art of organic synthesis.

One skilled in the art will recognize that the synthesis of the compounds of Formula (I) may require the need for the protection of certain functional groups (i.e., derivatization for the purpose of chemical compatibility with a particular reaction condition). Suitable protecting groups for the various functional groups of the compounds of formula (I) and methods for their installation and removal may be found in Greene et al., Protective Groups in Organic Synthesis, Wiley-Interscience, New York, (1999).

### EXAMPLES

The following examples exemplify illustrative examples of compounds of the present invention and are not to be construed as limiting the scope of the disclosure. Alternative mechanistic pathways and analogous structures within the scope of the invention may be apparent to those skilled in the art.

### General Methods

Solvents, reagents, and intermediates that are commercially available were used as received. Reagents and intermediates that are not commercially available were prepared in the manner described below

### Example 1

### Preparation of Intermediate Compound C

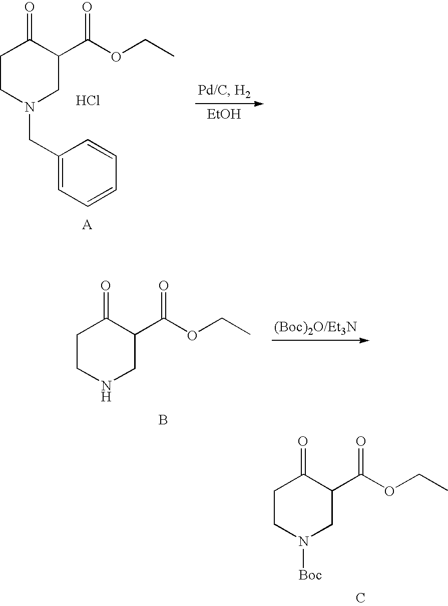

To a solution of 1-benzyl-2-carboethoxy-4-piperidone hydrochloride (A, 3.0 kg, 10.07 mol) in EtOH (30 L) was added Pd/C (300 g, 10% w/w). The resulting solution was put under H₂ atmosphere and stirred at room temperature at 4-5 bar for 3 hours. (Boc)₂O (2320 g) and Et₃N (1560 mL) were then added to the reaction mixture and the resulting solution was stirred at room temperature 1 hour, then filtered through a pad of Celite®. The filtrate was concentrated in vacuo and the resulting residue was dissolved in methylene chloride and water. The organic phase was separated, dried over MgSO₄ and concentrated in vacuo. The residue obtained was vacuum dried to provide compound C (2.5 kg, 91%).

### Example 2

### Preparation of Compound E

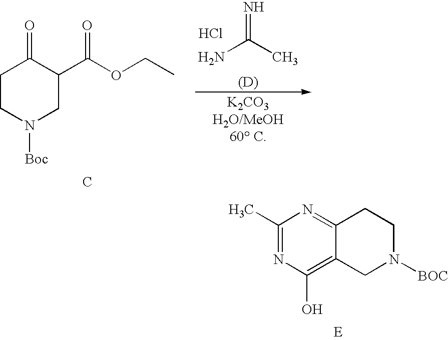

To a solution of acetamidine hydrochloride (D, 209 g, 2.21 mL) in a mixture of water (4000 mL) and methanol (1000 mL) was added potassium carbonate (370 g, 2.68 mol), followed by compound C (500 g, 1.84 mol). The mixture was heated to 60° C., allowed to stir at this temperature for about 15 hours, then cooled to room temperature. The reaction mixture was then neutralized using aqueous HCl (2N) and the resulting solution was diluted with dichloromethane (2000 mL). The organic phase was separated, dried over MgSO₄ and concentrated in vacuo. The residue obtained was triturated with n-Hexane and the precipitate formed was filtered, collected and dried under vacuum to provide compound E (293 g, 60%).

### Example 3

### A General Method for the Preparation of Tertiary Urea Libraries at R² of the Compounds of Formula (I)

To a solution of compound of formula H (0.025 mmol) in DCE/MeOH (25:1 v/v, 1 mL) is added a 0.5 M solution of an isocyanate compound of formula R^{a}—NCO (0.075 mmol) in DCE. The reaction mixture is then allowed to stir at room temperature for about 20 hours, after which time dichloroethane (0.5 mL), polystyrene isocyanate resin (0.057 g, 0.087 mmol) and polystyrene trisamine resin (0.049 g, 0.207 mmol) are added. The resultant reaction is allowed to stir at room temperature and monitored until complete. The reaction product is then filtered and the resin is washed with acetonitrile (0.5 mL). The filtrate and washings are combined and concentrated in vacuo to provide a compound of formula (I), wherein the R² group forms a tertiary urea with the nitrogen atom to which it is attached.

### Example 4

### A General Method for the Preparation of Amide Libraries at R² of the Compounds of Formula (I)

To a mixture of polystyrene EDC resin (0.106 g, 0.146 mmol) and a compound of formula H (0.025 mmol) in MeCN/THF (3:1 v/v, 1 mL) is added a 1 M solution of a carboxylic acid of formula R^{a}—COOH (0.038 mmol) in DMF. To the resulting mixture is then added a solution of HOBt (0.5M, 0.038 mmol) in MeCN/THF (3:1 v/v, 0.20 mL). The reaction is allowed to stir at room temperature for about 20 hours, after which time acetonitrile (0.5 mL), polystyrene isocyanate resin (0.049 g, 0.075 mmol) and polystyrene trisamine resin (0.035 g, 0.148 mmol) are added. The resultant reaction mixture is then allowed to stir at room temperature and monitored until completion. When complete, the reaction mixture is filtered, the resin is washed with acetonitrile (0.5 mL), and the combined filtrate and washing is concentrated in vacuo to provide a compound of formula (I), wherein the R² group forms an amide with the nitrogen atom to which it is attached.

### Example 5

### A General Method for the Preparation of N-Alkyl Compound Libraries at R² of the Compounds of Formula (I)

To a solution of a compound of formula H (0.025 mmol) in DMF/THF (1:1 v/v, 1 mL) is added a solution of an aldehyde of formula R^{a}—CHO (0.075 mmol) in DCE. To the resulting solution is added sodium triacetoxyborohydride (3 eq.) and the resulting reaction is allowed to stir at room temperature for about 20 hours. MeOH (0.5 mL) is then added to the reaction vessel and the vessel is shaken for 10 minutes or until gas evolution ceases. MP-TsOH resin (−100 mg) is then added to the reaction vessel, and the resultant mixture is shaken for about 2 hours, then filtered. The collected resin is then washed sequentially with DCE (3×) and methanol (3×). The washed resin is then diluted with 2N ammonia in methanol (1.5-2 mL) and the resulting solution is allowed to stir for about 1 hour, then filtered. The filtrate is concentrated in vacuo to provide a compound of formula (I), wherein the R² group is joined to the nitrogen atom to which it is attached via a CH₂— linker.

### Example 6

### GPR119Activation Assay

The ability of the Tetrahydropyrido[4,3-d]Pyrimidinone Derivatives to activate GPR119 and stimulate increases in cAMP levels can be determined using the LANCET™ cAMP kit (Perkin Elmer). HEK293 cells expressing human GPR119 are maintained in culture flasks at 37° C./5% CO₂ in DMEM containing 10% fetal bovine serum, 100 U/ml Pen/Strep, and 0.5 mg/ml geneticin. The media is then changed to Optimem and cells are incubated for about 15 hours at 37° C./5% CO₂. The Optimem is then aspirated and the cells are removed from the flasks using room temperature Hank's balanced saline solution (HBSS). The cells are then pelleted using centrifugation (1300 rpm, 7 minutes, room temperature), and resuspended in stimulation buffer (MSS, 0.1% BSA, 5 mM HEPES, 15 μM R^{O}-20) at 2.5×10⁶ cells/mL. Alexa Fluor 647-anti cAMP antibody (1:100) is then added to the cell suspension and incubated for 30 minutes. Representative compound(s) of formula (I) (6 μl at 2× concentration) in stimulation buffer containing 2% DMSO are then added to white 384 well Matrix plates. Cell suspension mix (6 μl) is then added to each well and incubated with the compound of formula (I) for 30 minutes. A cAMP standard curve is also created in each assay according to the kit protocol. Standard concentrations of cAMP in stimulation buffer (6 μl) are added to white 384 well plates. Subsequently, 6 μl of 1:100 anti-cAMP antibody is added to each well. Following the 30 minute incubation period, 12 μl of detection mix (included in kit) is added to all wells and incubated for 2-3 hours at room temperature. Fluorescence can be detected on the plates using an Envision instrument. The level of cAMP in each well can then be determined by extrapolation from the cAMP standard curve.

### Example 7

### Oral Glucose Tolerance Test

The effects of the Tetrahydropyrido[4,3-d]Pyrimidinone Derivatives in the Oral Glucose Tolerance Test can be determined as follows:

Male C57Bl/6NCrl mice (6-8 week old) are fasted overnight and randomly dosed with either vehicle (20% hydroxypropyl-β-cyclodextrin) or a representative compound of the invention (at 3, 10 or 30 mg/kg) via oral gavage (n=8 mice/group). Glucose is administered to the animals 30 minutes post-dosing (3 g/kg p.o.). Blood glucose is measured prior to administration of test compound and glucose, and at 20 minutes after glucose administration using a hand-held glucometer (Ascensia Elite, Bayer).

### Uses of the Tetrahydropyrido[4,3-d]Pyrimidinone Derivatives

The Tetrahydropyrido[4,3-d]Pyrimidinone Derivatives are useful in human and veterinary medicine for treating or preventing a Condition in a patient. In accordance with the invention, the Tetrahydropyrido[4,3-d]Pyrimidinone Derivatives can be administered to a patient in need of treatment or prevention of a Condition.

### Treatment of Obesity and Obesity-Related Disorders

The Tetrahydropyrido[4,3-d]Pyrimidinone Derivatives can be useful for treating obesity or an obesity-related disorder. Accordingly, in one embodiment, the invention provides methods for treating obesity or an obesity-related disorder in a patient, wherein the method comprises administering to the patient an effective amount of one or more Tetrahydropyrido[4,3-d]Pyrimidinone Derivatives, or a pharmaceutically acceptable salt, solvate, ester or prodrug thereof.

### Treatment of Diabetes

The Tetrahydropyrido[4,3-d]Pyrimidinone Derivatives can be useful for treating diabetes in a patient. Accordingly, in one embodiment, the present invention provides a method for treating diabetes in a patient, comprising administering to the patient an effective amount of one or more Tetrahydropyrido[4,3-d]Pyrimidinone Derivatives.

Examples of diabetes treatable or preventable using the Tetrahydropyrido[4,3-d]Pyrimidinone Derivatives include, but are not limited to, type I diabetes (insulin-dependent diabetes mellitus), type II diabetes (non-insulin dependent diabetes mellitus), idiopathic type I diabetes (Type Ib), latent autoimmumne diabetes in adults, early-onset type 2 diabetes (EOD), youth-onset atypical diabetes (YOAD), maturity onset diabetes of the young (MODY), malnutrition-related diabetes, gestational diabetes, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), type A insulin resistance syndrome, type B insulin resistance syndrome, lipatrophic diabetes and diabetes induced by n-cell toxins.

### Treatment of a Diabetic Complication

The Tetrahydropyrido[4,3-d]Pyrimidinone Derivatives can be useful for treating a diabetic complication in a patient. Accordingly, in one embodiment, the present invention provides a method for treating a diabetic complication in a patient, comprising administering to the patient an effective amount of one or more Tetrahydropyrido[4,3-d]Pyrimidinone Derivatives.

Examples of diabetic complications treatable or preventable using the Tetrahydropyrido[4,3-d]Pyrimidinone Derivatives include, but are not limited to, diabetic cataract, glaucoma, retinopathy, aneuropathy (such as diabetic neuropathy, polyneuropathy, mononeuropathy, autonomic neuropathy, microaluminuria and progressive diabetic neuropathyl), nephropathy, gangrene of the feet, immune-complex vasculitis, systemic lupsus erythematosus (SLE), atherosclerotic coronary arterial disease, peripheral arterial disease, nonketotic hyperglycemic-hyperosmolar coma, foot ulcers, joint problems, a skin or mucous membrane complication (such as an infection, a shin spot, a candidal infection or necrobiosis lipoidica diabeticorumobesity), hyperlipidemia, cataract, hypertension, syndrome of insulin resistance, coronary artery disease, a fungal infection, a bacterial infection, and cardiomyopathy.

### Treatment of a Metabolic Disorder

The Tetrahydropyrido[4,3-d]Pyrimidinone Derivatives can be useful for treating a metabolic disorder. Accordingly, in one embodiment, the invention provides methods for treating a metabolic disorder in a patient, wherein the method comprises administering to the patient an effective amount of one or more Tetrahydropyrido[4,3-d]Pyrimidinone Derivatives, or a pharmaceutically acceptable salt, solvate, ester or prodrug thereof.

Examples of metabolic disorders treatable include, but are not limited to, metabolic syndrome (also known as “Syndrome X”), impaired glucose tolerance, impaired fasting glucose, hypercholesterolemia, hyperlipidemia, hypertriglyceridemia, low HDL levels, hypertension, phenylketonuria, post-prandial lipidemia, a glycogen-storage disease, Gaucher's Disease, Tay-Sachs Disease, Niemann-Pick Disease, ketosis and acidosis.

In one embodiment, the metabolic disorder is hypercholesterolemia.

In another embodiment, the metabolic disorder is hyperlipidemia.

In another embodiment, the metabolic disorder is hypertriglyceridemia.

In still another embodiment, the metabolic disorder is metabolic syndrome.

In a further embodiment, the metabolic disorder is low HDL levels.

### Treatment of a Cardiovascular Disease

The Tetrahydropyrido[4,3-d]Pyrimidinone Derivatives can be useful for treating a cardiovascular disease. Accordingly, in one embodiment, the invention provides methods for treating a cardiovascular disease in a patient, wherein the method comprises administering to the patient an effective amount of one or more Tetrahydropyrido[4,3-d]Pyrimidinone Derivatives, or a pharmaceutically acceptable salt, solvate, ester or prodrug thereof.

Examples of cardiovascular diseases treatable or preventable using the present methods include, but are not limited to, atherosclerosis, congestive heart failure, circulatory shock, coronary artery disease, left ventricular hypertrophy, angina pectoris, cardiomyopathy, myocardial infarction and a cardiac arrhythmia.

In one embodiment, the cardiovascular disease is atherosclerosis.

In another embodiment, the cardiovascular disease is congestive heart failure.

### Combination Therapy

In one embodiment, the present invention provides methods for treating a Condition in a patient, the method comprising administering to the patient one or more Tetrahydropyrido[4,3-d]Pyrimidinone Derivatives, or a pharmaceutically acceptable salt, solvate, ester or prodrug thereof and at least one additional therapeutic agent that is not a Tetrahydropyrido[4,3-d]Pyrimidinone Derivative, wherein the amounts administered are together effective to treat or prevent a Condition.

Non-limiting examples of additional therapeutic agents useful in the present methods for treating or preventing a Condition include, anti-obesity agents, antidiabetic agents, any agent useful for treating metabolic syndrome, any agent useful for treating a cardiovascular disease, cholesterol biosynthesis inhibitors, cholesterol absorption inhibitors, bile acid sequestrants, probucol derivatives, IBAT inhibitors, nicotinic acid receptor (NAR) agonists, ACAT inhibitors, cholesteryl ester transfer proten (CETP) inhibitors, low-denisity lipoprotein (LDL) activators, fish oil, water-soluble fibers, plant sterols, plant stanols, fatty acid esters of plant stanols, or any combination of two or more of these additional therapeutic agents.

Non-limiting examples of anti-obesity agents useful in the present methods for treating a Condition include CB1 antagonists or inverse agonists such as rimonabant, neuropeptide γ antagonists, MCR4 agonists, MCH receptor antagonists, histamine H₃ receptor antagonists or inverse agonists, metabolic rate enhancers, nutrient absorption inhibitors, leptin, appetite suppressants and lipase inhibitors.

Non-limiting examples of appetite suppressant agents useful in the present methods for treating or preventing a Condition include cannabinoid receptor 1 (CB₁) antagonists or inverse agonists (e.g., rimonabant); Neuropeptide Y (NPY1, NPY2, NPY4 and NPY5) antagonists; metabotropic glutamate subtype 5 receptor (mGluR5) antagonists (e.g., 2-methyl-6-(phenylethynyl)-pyridine and 3[(2-methyl-1,4-thiazol-4-ypethynyl]pyridine); melanin-concentrating hormone receptor (MCH1R and MCH2R) antagonists; melanocortin receptor agonists (e.g., Melanotan-11 and Mc4r agonists); serotonin uptake inhibitors (e.g., dexfenfluramine and fluoxetine); serotonin (5HT) transport inhibitors (e.g., paroxetine, fluoxetine, fenfluramine, fluvoxamine, sertaline and imipramine); norepinephrine (NE) transporter inhibitors (e.g., desipramine, talsupram and nomifensine); ghrelin antagonists; leptin or derivatives thereof; opioid antagonists (e.g., nalmefene, 3-methoxynaltrexone, naloxone and nalterxone); orexin antagonists; bombesin receptor subtype 3 (BRS3) agonists; Cholecystokinin-A (CCK-A) agonists; ciliary neurotrophic factor (CNTF) or derivatives thereof (e.g., butabindide and axokine); monoamine reuptake inhibitors (e.g., sibutramine); glucagon-like peptide 1 (GLP-1) agonists; topiramate; and phytopharm compound 57.

Non-limiting examples of metabolic rate enhancers useful in the present methods for treating or preventing a Condition include acetyl-CoA carboxylase-2 (ACC2) inhibitors; beta adrenergic receptor 3 (P3) agonists; diacylglycerol acyltransferase inhibitors (DGAT1 and DGAT2); fatty acid synthase (FAS) inhibitors (e.g., Cerulenin); phosphodiesterase (PDE) inhibitors (e.g., theophylline, pentoxifylline, zaprinast, sildenafil, aminone, milrinone, cilostamide, rolipram and cilomilast); thyroid hormone p agonists; uncoupling protein activators (UCP-1, 2 or 3) (e.g., phytanic acid, 4-[(E)-2-(5,6,7,8-tetramethyl-2-naphthalenyl)-1-propenyl]benzoic acid and retinoic acid); acyl-estrogens (e.g., oleoyl-estrone); glucocorticoid antagonists; 11-beta hydroxy steroid dehydrogenase type 1 (11 (3 HSD-1) inhibitors; melanocortin-3 receptor (Mc3r) agonists; and stearoyl-CoA desaturase-1 (SCD-1) compounds.

Non-limiting examples of nutrient absorption inhibitors useful in the present methods for treating or preventing a Condition include lipase inhibitors (e.g., orlistat, lipstatin, tetrahydrolipstatin, teasaponin and diethylumbelliferyl phosphate); fatty acid transporter inhibitors; dicarboxylate transporter inhibitors; glucose transporter inhibitors; and phosphate transporter inhibitors.

Non-limiting examples of cholesterol biosynthesis inhibitors useful in the present methods for treating or preventing a Condition include HMG-CoA reductase inhibitors, squalene synthase inhibitors, squalene epoxidase inhibitors, and mixtures thereof.

Non-limiting examples of cholesterol absorption inhibitors useful in the present methods for treating or preventing a Condition include ezetimibe. In one embodiment, the cholesterol absorption inhibitor is ezetimibe.

HMG-CoA reductase inhibitors useful in the present methods for treating or preventing a Condition include, but are not limited to, statins such as lovastatin, pravastatin, fluvastatin, simvastatin, atorvastatin, cerivastatin, CI-981, resuvastatin, rivastatin, pitavastatin, rosuvastatin or L-659,699 ((E,E)-[3′R-(hydroxy-methyl)-4′-oxo-2′R-oxetanyl]-3,5,7R-trimethyl-2,4-undecadienoic acid).

Squalene synthesis inhibitors useful in the present methods for treating or preventing a Condition include, but are not limited to, squalene synthetase inhibitors; squalestatin 1; and squalene epoxidase inhibitors, such as NB-598 ((E)-N-ethyl-N-(6,6-dimethyl-2-hepten-4-ynyl)-3-[(3,3′-bithiophen-5-yl)methoxy]benzene-methanamine hydrochloride).

Bile acid sequestrants useful in the present methods for treating or preventing a Condition include, but are not limited to, cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof. Suitable inorganic cholesterol sequestrants include bismuth salicylate plus montmorillonite clay, aluminum hydroxide and calcium carbonate antacids.

Probucol derivatives useful in the present methods for treating or preventing a Condition include, but are not limited to, AGI-1067 and others disclosed in U.S. Pat. Nos. 6,121,319 and 6,147,250.

IBAT inhibitors useful in the present methods for treating or preventing a Condition include, but are not limited to, benzothiepines such as therapeutic compounds comprising a 2,3,4,5-tetrahydro-1-benzothiepine 1,1-dioxide structure such as are disclosed in International Publication No. WO 00/38727.

Nicotinic acid receptor agonists useful in the present methods for treating or preventing a Condition include, but are not limited to, those having a pyridine-3-carboxylate structure or a pyrazine-2-carboxylate structure, including acid forms, salts, esters, zwitterions and tautomers, where available. Other examples of nicotinic acid receptor agonists useful in the present methods include nicotinic acid, niceritrol, nicofuranose and acipimox. An example of a suitable nicotinic acid product is NIASPAN® (niacin extended-release tablets) which are available from Kos Pharmaceuticals, Inc. (Cranbury, N.J.). Further nicotinic acid receptor agonists useful in the present methods for treating or preventing a Condition include, but are not limited to, the compounds disclosed in U.S. Patent Publication Nos. 2006/0264489 and 2007/0066630, and U.S. patent application Ser. No. 11/771,538, each of which is incorporated herein by reference.

ACAT inhibitors useful in the present methods for treating or preventing a Condition include, but are not limited to, avasimibe, HL-004, lecimibide and CL-277082 (N-(2,4-difluorophenyl)-N—[[4-(2,2-dimethylpropyl)phenyl]-methyl]-N-heptylurea). See P. Chang et al., “Current, New and Future Treatments in Dyslipidaemia and Atherosclerosis”, Drugs 2000 July; 60(1); 55-93, which is incorporated by reference herein.

CETP inhibitors useful in the present methods for treating or preventing a Condition include, but are not limited to, those disclosed in International Publication No. WO 00/38721 and U.S. Pat. No. 6,147,090, which are incorporated herein by reference.

LDL-receptor activators useful in the present methods for treating or preventing a Condition include, but are not limited to, include HOE-402, an imidazolidinyl-pyrimidine derivative that directly stimulates LDL receptor activity. See M. Huettinger et al., “Hypolipidemic activity of HOE-402 is Mediated by Stimulation of the LDL Receptor Pathway”, Arterioscler. Thromb. 1993; 13:1005-12.

Natural water-soluble fibers useful in the present methods for treating or preventing a Condition include, but are not limited to, psyllium, guar, oat and pectin.

Fatty acid esters of plant stanols useful in the present methods for treating or preventing a Condition include, but are not limited to, the sitostanol ester used in BENECOL® margarine.

Non-limiting examples of antidiabetic agents useful in the present methods for treating a Condition include insulin sensitizers, β-glucosidase inhibitors, DPP-IV inhibitors, insulin secretagogues, hepatic glucose output lowering compounds, antihypertensive agents, sodium glucose uptake transporter 2 (SGLT-2) inhibitors, insulin and insulin-containing compositions, and anti-obesity agents as set forth above.

In one embodiment, the antidiabetic agent is an insulin secretagogue. In one embodiment, the insulin secretagogue is a sulfonylurea.

Non-limiting examples of sulfonylureas useful in the present methods include glipizide, tolbutamide, glyburide, glimepiride, chlorpropamide, acetohexamide, gliamilide, gliclazide, gliquidone, glibenclamide and tolazamide.

In another embodiment, the insulin secretagogue is a meglitinide.

Non-limiting examples of meglitinides useful in the present methods for treating a Condition include repaglinide, mitiglinide, and nateglinide.

In still another embodiment, the insulin secretagogue is GLP-1 or a GLP-1 mimetic.

Non-limiting examples of GLP-1 mimetics useful in the present methods include Byetta-Exanatide, Liraglutinide, CJC-1131 (ConjuChem, Exanatide-LAR (Amylin), BIM-51077 (Ipsen/LaRoche), ZP-10 (Zealand Pharmaceuticals), and compounds disclosed in International Publication No. WO 00/07617.

Other non-limiting examples of insulin secretagogues useful in the present methods include exendin, GIP and secretin.

In another embodiment, the antidiabetic agent is an insulin sensitizer.

Non-limiting examples of insulin sensitizers useful in the present methods include PPAR activators or agonists, such as troglitazone, rosiglitazone, pioglitazone and englitazone; biguanidines such as metformin and phenformin; PTP-1B inhibitors; and glucokinase activators.

In another embodiment, the antidiabetic agent is a (3-Glucosidase inhibitor.

Non-limiting examples of β-Glucosidase inhibitors useful the present methods include miglitol, acarbose, and voglibose.

In another embodiment, the antidiabetic agent is an hepatic glucose output lowering agent.

Non-limiting examples of hepatic glucose output lowering agents useful in the present methods include Glucophage and Glucophage XR.

In yet another embodiment, the antidiabetic agent is insulin, including all formulations of insulin, such as long acting and short acting forms of insulin.

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In another embodiment, the antidiabetic agent is a DPP-IV inhibitor.

Non-limiting examples of DPP-IV inhibitors useful in the present methods include sitagliptin, saxagliptin (Januvia™, Merck), denagliptin, vildagliptin (Galvus™, Novartis), alogliptin, alogliptin benzoate, ABT-279 and ABT-341 (Abbott), ALS-2-0426 (Alantos), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck).

In a further embodiment, the antidiabetic agent is a SGLT-2 inhibitor.

Non-limiting examples of SGLT-2 inhibitors useful in the present methods include dapagliflozin and sergliflozin, AVE2268 (Sanofi-Aventis) and T-1095 (Tanabe Seiyaku).

Non-limiting examples of antihypertensive agents useful in the present methods for treating a Condition include (3-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

In one embodiment, the antidiabetic agent is an agent that slows or blocks the breakdown of starches and certain sugars.

Non-limiting examples of antidiabetic agents that slow or block the breakdown of starches and certain sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in International Publication No. WO 00/07617.

Other specific additional therapeutic agents useful in the present methods for treating or preventing a Condition include, but are not limited to, rimonabant, 2-methyl-6-(phenylethynyl)-pyridine, 3[(2-methyl-1,4-thiazol-4-yl)ethynyl]pyridine, Melanotan-H, dexfenfluramine, fluoxetine, paroxetine, fenfluramine, fluvoxamine, sertaline, imipramine, desipramine, talsupram, nomifensine, leptin, nalmefene, 3-methoxynaltrexone, naloxone, nalterxone, butabindide, axokine, sibutramine, topiramate, phytopharm compound 57, Cerulenin, theophylline, pentoxifylline, zaprinast, sildenafil, aminone, milrinone, cilostamide, rolipram, cilomilast, phytanic acid, 4-[(E)-2-(5,6,7,8-tetramethyl-2-naphthalenyl)-1-propenyl]benzoic acid, retinoic acid, oleoyl-estrone, orlistat, lipstatin, tetrahydrolipstatin, teasaponin and diethylumbelliferyl phosphate.

In one embodiment, the present combination therapies for treating or preventing diabetes comprise administering a compound of formula (I), an antidiabetic agent and/or an antiobesity agent.

In another embodiment, the present combination therapies for treating or preventing diabetes comprise administering a compound of formula (I) and an antidiabetic agent.

In another embodiment, the present combination therapies for treating or preventing diabetes comprise administering a compound of formula (I) and an anti-obesity agent.

In one embodiment, the present combination therapies for treating or preventing obesity comprise administering a compound of formula (I), an antidiabetic agent and/or an antiobesity agent.

In another embodiment, the present combination therapies for treating or preventing obesity comprise administering a compound of formula (I) and an antidiabetic agent.

In another embodiment, the present combination therapies for treating or preventing obesity comprise administering a compound of formula (I) and an anti-obesity agent.

In one embodiment, the present combination therapies for treating or preventing metabolic syndrome comprise administering a compound of formula (I) and one or more additional therapeutic agents selected from: anti-obesity agents, antidiabetic agents, any agent useful for treating metabolic syndrome, any agent useful for treating a cardiovascular disease, cholesterol biosynthesis inhibitors, sterol absorption inhibitors, bile acid sequestrants, probucol derivatives, IBAT inhibitors, nicotinic acid receptor (NAR) agonists, ACAT inhibitors, cholesteryl ester transfer protein (CETP) inhibitors, low-density lipoprotein (LDL) activators, fish oil, water-soluble fibers, plant sterols, plant stanols and fatty acid esters of plant stanols.

In one embodiment, the additional therapeutic agent is a cholesterol biosynthesis inhibitor. In another embodiment, the cholesterol biosynthesis inhibitor is a squalene synthetase inhibitor. In another embodiment, the cholesterol biosynthesis inhibitor is a squalene epoxidase inhibitor. In still another embodiment, the cholesterol biosynthesis inhibitor is an HMG-CoA reductase inhibitor. In another embodiment, the HMG-CoA reductase inhibitor is a statin. In yet another embodiment, the statin is lovastatin, pravastatin, simvastatin or atorvastatin.

In one embodiment, the additional therapeutic agent is a cholesterol absorption inhibitor. In another embodiment, the cholesterol absorption inhibitor is ezetimibe.

In one embodiment, the additional therapeutic agent comprises a cholesterol absorption inhibitor and a cholesterol biosynthesis inhibitor. In another embodiment, the additional therapeutic agent comprises a cholesterol absorption inhibitor and a statin. In another embodiment, the additional therapeutic agent comprises ezetimibe and a statin. In another embodiment, the additional therapeutic agent comprises ezetimibe and simvastatin.

In one embodiment, the present combination therapies for treating or preventing metabolic syndrome comprise administering a compound of formula (I), an antidiabetic agent and/or an antiobesity agent.

In another embodiment, the present combination therapies for treating or preventing metabolic syndrome comprise administering a compound of formula (I) and an antidiabetic agent.

In another embodiment, the present combination therapies for treating or preventing metabolic syndrome comprise administering a compound of formula (I) and an anti-obesity agent.

In one embodiment, the present combination therapies for treating or preventing a cardiovascular disease comprise administering one or more compounds of formula (I), and an additional agent useful for treating or preventing a cardiovascular disease.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Tetrahydropyrido[4,3-d]Pyrimidinone Derivatives are administered during a time when the additional therapeutic agent(s) exert their prophylactic or therapeutic effect, or vice versa.

In another embodiment, the one or more Tetrahydropyrido[4,3-d]Pyrimidinone Derivatives and the additional therapeutic agent(s) are administered in doses commonly employed when such agents are used as monotherapy for treating a Condition.

In another embodiment, the one or more Tetrahydropyrido[4,3-d]Pyrimidinone Derivatives and the additional therapeutic agent(s) are administered in doses lower than the doses commonly employed when such agents are used as monotherapy for treating a Condition.

In still another embodiment, the one or more Tetrahydropyrido[4,3-d]Pyrimidinone Derivatives and the additional therapeutic agent(s) act synergistically and are administered in doses lower than the doses commonly employed when such agents are used as monotherapy for treating a Condition.

In one embodiment, the one or more Tetrahydropyrido[4,3-d]Pyrimidinone Derivatives and the additional therapeutic agent(s) are present in the same composition. In one embodiment, this composition is suitable for oral administration. In another embodiment, this composition is suitable for intravenous administration.

The one or more Tetrahydropyrido[4,3-d]Pyrimidinone Derivatives and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Tetrahydropyrido[4,3-d]Pyrimidinone Derivatives and the additional therapeutic agent(s) may inhibit the resistance of a Condition to these agents.

In one embodiment, when the patient is treated for diabetes or a diabetic complication, the additional therapeutic agent is an antidiabetic agent which is not a Tetrahydropyrido[4,3-d]Pyrimidinone Derivative. In another embodiment, the additional therapeutic agent is an agent useful for reducing any potential side effect of a Tetrahydropyrido[4,3-d]Pyrimidinone Derivative. Such potential side effects include, but are not limited to, nausea, vomiting, headache, fever, lethargy, muscle aches, diarrhea, general pain, and pain at an injection site.

In one embodiment, the additional therapeutic agent is used at its known therapeutically effective dose. In another embodiment, the additional therapeutic agent is used at its normally prescribed dosage. In another embodiment, the additional therapeutic agent is used at less than its normally prescribed dosage or its known therapeutically effective dose.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Tetrahydropyrido[4,3-d]Pyrimidinone Derivative(s) and the other agent(s) for treating diseases or conditions listed above can be administered simultaneously or sequentially. This particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the one or more Tetrahydropyrido[4,3-d]Pyrimidinone Derivatives and the additional therapeutic agent(s)can when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

In one embodiment, the invention provides compositions comprising an effective amount of one or more compounds of formula (I) or a pharmaceutically acceptable salt, solvate, ester or prodrug thereof, and a pharmaceutically acceptable carrier.

For preparing pharmaceutical compositions from the compounds of formula (I), inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, the Tetrahydropyrido[4,3-d]Pyrimidinone Derivative is administered orally.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

When the invention comprises a combination of one or more Tetrahydropyrido[4,3-d]Pyrimidinone Derivatives and an additional therapeutic agent, the two active components may be co-administered simultaneously or sequentially, or a single pharmaceutical composition comprising one or more Tetrahydropyrido[4,3-d]Pyrimidinone Derivatives and an additional therapeutic agent in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of the advantageous effect of the combination.

In one embodiment, the components of a combination therapy regime are to be administered simultaneously, they can be administered in a single composition with a pharmaceutically acceptable carrier.

In another embodiment, when the components of a combination therapy regime are to be administered separately or sequentially, they can be administered in separate compositions, each containing a pharmaceutically acceptable carrier.

The components of the combination therapy can be administered individually or together in any conventional dosage form such as capsule, tablet, powder, cachet, suspension, solution, suppository, nasal spray, etc.

### Kits

In one aspect, the present invention provides a kit comprising an effective amount of one or more Compounds of Formula (I), or a pharmaceutically acceptable salt or solvate of the compound and a pharmaceutically acceptable carrier, vehicle or diluent.

In another aspect the present invention provides a kit comprising an amount of one or more Tetrahydropyrido[4,3-d]Pyrimidinone Derivatives, or a pharmaceutically acceptable salt or solvate of the compound and an amount of at least one additional therapeutic agent listed above, wherein the combined amounts are effective for treating or preventing diabetes, a diabetic complication impaired glucose tolerance or impaired fasting glucosein a patient.

When the components of a combination therapy regime are to be administered in more than one composition, they can be provided in a kit comprising in a single package, one or more containers, each comprising one or more Tetrahydropyrido[4,3-d]Pyrimidinone Derivatives in a pharmaceutically acceptable carrier, and a separate container comprising an additional therapeutic agent in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A method for treating diabetes in a patient, the method comprising administering to the patient an effective amount of one or more compounds having the formula: 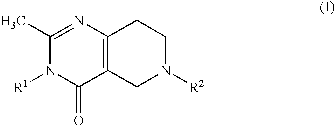
or a pharmaceutically acceptable salt, solvate, ester or prodrug thereof, wherein R¹ and R² are as defined above in the specification.

2. A method for treating obesity in a patient, the method comprising administering to the patient an effective amount of one or more compounds having the formula: 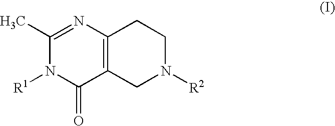
or a pharmaceutically acceptable salt, solvate, ester or prodrug thereof, wherein R¹ and R² are as defined above in the specification.

3. A method for treating metabolic syndrome in a patient, the method comprising administering to the patient an effective amount of one or more compounds having the formula: 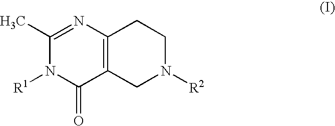
or a pharmaceutically acceptable salt, solvate, ester or prodrug thereof, wherein R¹ and R² are as defined above in the specification.

4. The method of claim 1, further comprising administering to the patient at least one antidiabetic agent and/or at least one antiobesity agent that is not a compound of claim 1, and wherein the amounts administered are together effective to treat diabetes.

5. The method of claim 2, further comprising administering to the patient at least one antiobesity agent that is different from the compounds of claim 1, and wherein the amounts administered are together effective to treat obesity.

6. The method of claim 3, further comprising administering to the patient at least one antidiabetic agent and/or at least one antiobesity agent that is different from the compounds of claim 1, and wherein the amounts administered are together effective to treat metabolic syndrome.

7. The method of claim 4, comprising administering at least one antidiabetic agent that is different from the compounds of claim 1.

8. The method of claim 7, wherein the antidiabetic agent is an insulin sensitizer, a β-glucosidase inhibitor, a DPP-IV inhibitor, an insulin secretagogue, an hepatic glucose output lowering compound, an antihypertensive agent, a sodium glucose uptake transporter 2 (SGLT-2) inhibitor, insulin, an insulin-containing composition, and an antiobesity agent.

9-20. (canceled)

21. The method of claim 4, comprising administering at least one antiobesity agent that is different from the compounds of claim 1.

22. The method of claim 21, wherein the antiobesity agent is a neuropeptide γ antagonist, an MCR4 agonist, an MCH receptor antagonist, a protein hormone, an AMP kinase activator, a CB1 antagonist, a GLP-1 agonist or a lipase inhibitor.

23. (canceled)

24. The method of claim 1, wherein the diabetes is type I diabetes.

25. The method of claim 1, wherein the diabetes is type II diabetes.

26. The method of claim 5, wherein the antiobesity agent is a neuropeptide γ antagonist, an MCR4 agonist, an MCH receptor antagonist, a protein hormone, an AMP kinase activator, a CB 1 antagonist, a GLP-1 agonist or a lipase inhibitor.

27. The method of claim 26, wherein the antiobesity agent is orlistat, leptin, or adiponectin.

28. The method of claim 6, comprising administering at least one antidiabetic agent that is different from the compounds of claim 1.

29. The method of claim 28, wherein the antidiabetic agent is an insulin sensitizer, a β-glucosidase inhibitor, a DPP-IV inhibitor, an insulin secretagogue, an hepatic glucose output lowering compound, an antihypertensive agent, a sodium glucose uptake transporter 2 (SGLT-2) inhibitor, insulin, an insulin-containing composition, and an antiobesity agent.

30-41. (canceled)

42. The method of claim 6, comprising administering at least one antiobesity agent that is different from the compounds of claim 1.

43. The method of claim 42, wherein the antiobesity agent is a neuropeptide γ antagonist, an MCR4 agonist, an MCH receptor antagonist, a protein hormone, an AMP kinase activator, a CB1 antagonist, a GLP-1 agonist or a lipase inhibitor.

44. (canceled)
